(19) United States
(12) Patent Application Publication
(10) Pub. No.: US 2010/0317084 A1
(43) Pub. Date: Dec. 16, 2010

(54) RECOMBINANT BACTERIA COMPRISING VECTORS FOR EXPRESSION OF NUCLEIC ACID SEQUENCES ENCODING ANTIGENS

(75) Inventor: Roy Curtiss, III, Paradise Valley, AZ (US)

Correspondence Address: POLSINELLI SHUGHART PC, 700 W. 47TH STREET, SUITE 1000, KANSAS CITY, MO, 64112-1802, US

(73) Assignees: THE Arizona Board of Regents for and on behalf of Arizona State University, Tempe, AZ (US); The Washington University, St. Louis, MO (US)

(21) Appl. No.: 12/599,655

(22) PCT Filed: May 9, 2008
(86) PCT No.: PCT/US08/63303

§ 371 Date: Aug. 20, 2010

Related U.S. Application Data
(60) Provisional application No. 60/917,306, filed on May 10, 2007.

(51) Int. Cl. C12N 1/21

(52) U.S. Cl. 435/252.3

(57) ABSTRACT

The invention encompasses a recombinant bacterium that comprises at least one vector capable of expressing a nucleic acid sequence encoding an antigen. In particular, the bacterium comprises at least one chromosomally encoded essential nucleic acid that is altered so that it is not expressed, and at least one extrachromosomal vector.

### GOVERNMENTAL RIGHTS

This invention was made with government support under 5R01DE006669, R01A1056289, and R01A160557 awarded by the National Institutes of Health. The government has certain rights in the invention.

### FIELD OF THE INVENTION

The invention encompasses a recombinant bacterium that comprises at least one vector capable of expressing a nucleic acid sequence encoding an antigen.

### BACKGROUND OF THE INVENTION

Recombinant microorganisms have widespread utility and importance. One important use of these microorganisms is as live vaccines to produce an immune response. When the recombinant microorganism is to be utilized as a live vaccine for vertebrate, certain considerations must be taken into account. To provide a benefit beyond that of a nonliving vaccine, the live vaccine microorganism must attach to, invade, and survive in lymphoid tissues of the vertebrate and expose these immune effector sites in the vertebrate to antigen for an extended period of time. By this continual stimulation, the vertebrate's immune system becomes more highly reactive to the antigen than that provided by a nonliving vaccine. Therefore, preferred live vaccines are attenuated pathogens of the vertebrate, particularly pathogens that colonize the gut-associated lymphoid tissue (GALT), nasal associated lymphoid tissue (NALT), or bronchial-associated lymphoid tissue (BALT). An additional advantage of these attenuated pathogens over nonliving vaccines is that these pathogens have elaborate mechanisms to gain access to lymphoid tissues, and thus efficient exposure to the vertebrate's immune system can be expected. In contrast, nonliving vaccines will only provide an immune stimulus if the vaccine is passively exposed to the immune system, or if host mechanisms bring the vaccine to the immune system.

However, delivery of a single protective antigen to a host does not necessarily induce protective immunity against the pathogen from which the antigen was derived, since not all individuals are identical or able to mount immune responses against all potential protective antigens. As such, it would be preferable for a single recombinant bacterial strain to be able to express and deliver multiple different protective antigens derived from a given pathogen to ensure that all individuals immunized will at least be able to mount a protective immune response against at least one of the expressed protective antigens. Such a vaccine design requires the use of multiple vectors, and this in turn has the potential to lead to genetic instability that would not be acceptable to regulatory agencies charged with ensuring the consistency of vaccine products delivered for use to immunize agriculturally important animals, companion animals and especially humans.

Consequently, there is a need in the art for a recombinant bacterium capable of delivering multiple antigens to a host in a system with little to no genetic instability.

### SUMMARY OF THE INVENTION

Accordingly, the present invention encompasses a recombinant bacterium. The bacterium comprises a first chromosomally encoded essential nucleic acid sequence, wherein the first essential nucleic acid sequence is altered so that it is not expressed and a second chromosomally encoded essential nucleic acid sequence, wherein the second essential nucleic acid sequence is altered so that it is not expressed. The bacterium further comprises a first extrachromosomal vector, the vector comprising a nucleic acid sequence, that when expressed, substantially functions as the first essential nucleic acid sequence, and a second extrachromosomal vector, the vector comprising a nucleic acid sequence, that when expressed, substantially functions as the second essential nucleic acid sequence.

Another aspect of the invention comprises a recombinant bacterium. The bacterium comprises a chromosomally encoded essential nucleic acid sequence whose expression is necessary for a metabolic activity essential for virulence, wherein the essential nucleic acid sequence is altered so that it is not expressed, and an extrachromosomal vector, the vector comprising a nucleic acid sequence, that when expressed, substantially functions as the essential nucleic acid sequence.

Other aspects and iterations of the invention are described more thoroughly below.

### BRIEF DESCRIPTION OF THE FIGURES

FIG. 1 depicts a schematic that illustrates the use of an extra-chromosomal vector encoding aspartate semialdehyde dehydrogenase (Asd), an enzyme essential for the synthesis of diaminopimelic acid (DAP), for complementing a bacterium that comprises a Δasd mutation. The extra-chromosomal vector also encodes at least one foreign nucleic acid sequence (genX) that can specify an antigen of interest, such as a protective antigen.

FIG. 2 depicts a schematic for different Asd⁺ vectors comprising pSC101, p15A, pBR, and pUC origins of replication.

FIG. 3 depicts a schematic for different DadB⁺ vectors comprising the P22 P_{L} promoter.

FIG. 4 depicts a MurA⁺ vector that may be used for complementing a bacterium, such as a Salmonella bacterium, comprising the chromosomal deletion-insertion mutation ΔP_{murA}::TT araC P_{BAD} murA. The schematic depicts the deletion of 41 by between murA and yrbA and the insertion of the more tightly regulated P_{BAD} araC::TT with ATG-murA. Such a vector may further be used to express at least one nucleic acid sequence encoding an antigen of interest.

FIG. 5 depicts a Murl⁺ vector that may be used for complementing a Δmurl bacterium, such as a Salmonella bacterium. The schematic depicts the deletion of 861 bp of the murl nucleic acid sequence (murl₅₈ to murl₊₃₉). Such a vector may further be used to express at least one nucleic acid sequence encoding an antigen of interest.

FIG. 6 depicts a plasmid system that may be used to translocate an overexpressed secreted antigen outside of the cell cytoplasm. The β-lactamase signal sequence peptide is located at the N-terminal end.

FIG. 7 depicts a plasmid system that may be used to translocate an overexpressed secreted antigen outside of the cell cytoplasm where the signal sequence peptide is located at the N-terminal end and posseses the C-terminal end of β-lactamase to further enhance secretion.

FIG. 8 depicts an alternate Type II secretion Asd⁺ vector that may be used for expressing a nucleic acid sequence encoding an antigen of interest which harbors the ompA signal sequence (ompA SS). The map of pYA4102 and the nucleotide sequences of the P_{trc} promoter region including the multi-cloning sites and the site of cleavage by the Salmonella signal peptidase are shown.

FIG. 9 depicts another alternate Type II secretion Asd⁺ vector that may be used for expressing a nucleic acid sequence encoding an antigen of interest which harbors the phoA signal sequence (phoA SS). The map of pYA4106 and the nucleotide sequences of the P_{trc} promoter region including the multi-cloning sites and site of cleavage by the Salmonella signal peptidase are shown.

FIG. 10 depicts a P_{asd}P_{sopE}-SopE-CFPIO-ESAT-6 Fusion Type III secretion vector that may be used for expressing a nucleic acid sequence encoding an antigen of interest. c2*: codon optimized with improved Shine-Dalgarno sequence

FIG. 11 depicts a schematic of the pYA3681 plasmid conferring a regulated lysis phenotype when in an appropriate bacterial host.

FIG. 12 depicts an AroA⁺ vector that may be used for complementing an aromatic amino acids and vitamin deficient ΔaroA bacterium, such as a Salmonella bacterium. Such a vector may further be used to express at least one nucleic acid sequence encoding an antigen of interest. The schematic depicts the deletion of 1296 by of aroA, including the SD region and 1284 by of aroA (aroA₋₁₂ to aroAₗ₂₈₄).

FIG. 13 depicts an AroC⁺ vector that may be used for complementing an aromatic amino acids and vitamin deficient ΔaroC bacterium, such as a Salmonella bacterium. Such a vector may further be used to express at least one nucleic acid sequence encoding an antigen of interest. The schematic depicts the deletion of 1083 by of aroC (aroC₁ to aroC₁₀₈₃), leaving the stop codon TAA.

FIG. 14 depicts an AroD⁺ vector that may be used for complementing an aromatic amino acids and vitamin deficient ΔaroD bacterium, such as a Salmonella bacterium. Such a vector may further be used to express at least one nucleic acid sequence encoding an antigen of interest. The schematic depicts the deletion of 769 by of aroD, including the SD region and 759 by of aroD (aroD₋₁₀ to aroD₇₅₉).

FIG. 15 depicts an IlvC⁺ vector that may be used for complementing an essential isoleucine and valine amino acids deficient ΔilvC bacterium, such as a Salmonella bacterium. Such a vector may further be used to express at least one nucleic acid sequence encoding an antigen of interest. The schematic depicts the deletion of 1476 by of ilvC (ilvC₁ to ilvC₁₄₇₆).

FIG. 16 depicts an IlvE⁺ vector that may be used for complementing an essential isoleucine and valine amino acids deficient ΔilvE bacterium, such as a Salmonella bacterium. Such a vector may further be used to express at least one nucleic acid sequence encoding an antigen of interest. The schematic depicts the deletion of 940 by of ilvE, including the SD region and 930 by of ilvE (ilvE₋₁₀ to ilvE₉₃₀).

### DETAILED DESCRIPTION OF THE INVENTION

The present invention provides a recombinant bacterium comprising at least one chromosomally encoded essential nucleic acid sequence, wherein the essential nucleic acid sequence is altered so that it is not expressed, and at least one extrachromosomal vector. An “essential nucleic acid” is a native nucleic acid whose expression is necessary for cell viability or a metabolic activity essential for virulence. Consequently, a bacterium of the invention is non-viable and/or avirulent if an essential nucleic acid sequence is not expressed. Therefore, the bacterium of the invention further comprises at least one extrachromosomal vector. The vector comprises a nucleic acid sequence, that when expressed, substantially functions as the essential nucleic acid. Hence, the bacterium is viable and/or virulent when the vector is expressed. This promotes stable maintenance of the vector. Additionally, the vector may comprise a nucleic acid sequence encoding at least one antigen. This enables stable production of an antigen by the recombinant bacterium. In exemplary embodiments, the antigen elicits a protective immune response when a composition comprising the recombinant bacterium is administered to a host.

In each of the embodiments herein, the recombinant bacterium typically belongs to the Enterobaceteriaceae. The Enterobacteria family comprises species from the following genera: Alterococcus, Aquamonas, Aranicola, Arsenophonus, Brenneria, Budvicia, Buttiauxella, Candidatus Phlomobacter, Cedeceae, Citrobacter, Edwardsiella, Enterobacter, Erwinia, Escherichia, Ewingella, Hafnia, Klebsiella, Kluyvera, Leclercia, Leminorella, Moellerella, Morganella, Obesumbacterium, Pantoea, Pectobacterium, Photorhabdus, Plesiomonas, Pragia, Proteus, Providencia, Rahnella, Raoultella, Salmonella, Samsonia, Serratia, Shigella, Sodalis, Tatumella, Trabulsiella, Wigglesworthia, Xenorhbdus, Yersinia, Yokenella. In certain embodiments, the recombinant bacterium is typically a pathogenic species of the Enterobaceteriaceae. Due to their clinical significance, Escherichia coli, Shigella, Edwardsiella, Salmonella, Citrobacter, Klebsiella, Enterobacter, Serratia, Proteus, Morganella, Providencia and Yersinia are considered to be particularly useful. In other embodiments, the recombinant bacterium may be a species or strain commonly used for a vaccine.

Some embodiments of the instant invention comprise a species or subspecies of the Salmonella genera. For instance, the recombinant bacterium may be a Salmonella enterica serovar. In an exemplary embodiment, a bacterium of the invention may be derived from the S. enterica serovar, S. Typhimurium, S. Typhi, S. Paratyphi, S. Gallinarum, S. Enteritidis, S. Choleraesius, S. Arizona, or S. Dublin.

A recombinant bacterium of the invention derived from Salmonella may be particularly suited to use as a vaccine. Infection of a host with a Salmonella strain typically leads to colonization of the gut-associated lymphoid tissue (GALT) or Peyer's patches, which leads to the induction of a generalized mucosal immune response to the recombinant bacterium. Further penetration of the bacterium into the mesenteric lymph nodes, liver and spleen may augment the induction of systemic and cellular immune responses directed against the bacterium. Thus the use of recombinant Salmonella for oral immunization stimulates all three branches of the immune system, which is particularly important for immunizing against infectious disease agents that colonize on and/or invade through mucosal surfaces.

In an alternative embodiment, a bacterium of the invention may be a bacterium included in Table 1 below.

A recombinant bacterium of the invention, compositions comprising a recombinant bacterium, and methods of using a recombinant bacterium are described in more detail below.

TABLE 1
Bacterial Strains

| | |
|---|---|
| Strain | Genotype |

Escherichia coli

| | |
|---|---|
| χ6097 | F⁻ araD139 Δ(proAB-lac) λ⁻ φ80dlacZDM15 rpsL ΔasdA4 Δ(zhf-2::Tn10) thi-1 |
| χ6212 | F⁻ Δ(argF-lacZYA)-U169 glnV44 λ⁻ deoR φ80dlacZDM15 gyrA96 |
| | recA1 relA1 endA1 ΔasdA4 Δ(zhf-2::Tn10) thi-1 hsdR17 |
| χ7213 | thr-1 leuB6 fhuA21 lacY1 glnV44 recA1 ΔasdA4 Δ(zhf-2::Tn10) thi-1 |
| | RP4-2-Tc::Mu [λpir]; Km^{r} |
| χ7376 | F⁻ araD139 Δ(proAB-lac) λ⁻ φ80dlacZDM15 rpsL ΔasdA4 Δ(zhf- |
| | 2::Tn10) thi-1 Δalr ΔdadX |
| χ7377 | F⁻ mcrA Δ(mcrBC-hsdRMS-mrr) λ⁻ φ80dlacZΔM15 ΔlacX74 recA1 |
| | endA1 araD139 Δ(ara-leu)-7697 galU galK rpsL nupG Δalr ΔdadX |
| | ΔasdA4 |
| χ7395 | F⁻ Δ(argF-lacZYA)-U169 glnV44 λ⁻ deoR φ80dlacZDM15 gyrA96 |
| | recA1 relA1 endA1 ΔasdA4 Δ(zhf-2::Tn10) thi-1 hsdR17 Δalr ΔdadX |

Salmonella enterica Typhimurium UK-1

| | |
|---|---|
| χ3761 | UK-1 wild type |
| χ8133 | Δcya-27 Δcrp-27 ΔasdA16 |
| χ8276 | ΔasdA16 |
| χ8645 | ΔP_{murA7}::TT araC P_{BAD} murA |
| χ8660 | ΔP_{recA10}::TT araC P_{BAD} recA |
| χ8897 | ΔdadB4 |
| χ8898 | Δalr-3 |
| χ8901 | Δalr-3 ΔdadB4 |
| χ8937 | ΔrelA1123 ΔendA2311 ΔasdA19::TT araC P_{BAD} c2 |
| | ΔP_{murA7}::TT araC P_{BAD} murA Δ(gmd-fcl)-26 |
| χ8958 | ΔasdA33 |
| χ9040 | Δalr-3 ΔdadB4 ΔasdA16 |
| χ9048 | Δalr-3 ΔdadB4 ΔP_{phoPQ107}::TT araC P_{BAD} phoPQ ΔasdA18::TT araC |
| | P_{BAD} c2 |
| χ9052 | Δalr-3 ΔdadB4 ΔasdA33 |
| χ9070 | ΔrecF126 |
| χ9072 | ΔrecJ1315 |
| χ9081 | ΔrecJ1315 ΔrecF126 |
| χ9088 | Δpmi-2426 Δ(gmd-fcl)-26 ΔasdA16 ΔP_{fur33}::TT araC P_{BAD} fur |
| χ9241 | ΔpabA1516 ΔpabB232 ΔasdA16 ΔaraBAD23 ΔrelA198::araC P_{BAD} |
| | lacI TT |
| χ9291 | Δalr-3 ΔdadB4 ΔP_{phoPQ107}::TT araC P_{BAD} phoPQ ΔasdA18::TT araC |
| | P_{BAD} c2 ΔrecJ1315 ΔrecF126 |
| χ9292 | Δalr-3 ΔdadB4 ΔphoP233 ΔasdA18::TT araC P_{BAD} c2 ΔrecJ1315 |
| | ΔrecF126 |
| χ9340 | Δalr-3 ΔdadB4 ΔP_{phoPQ107}::TT araC P_{BAD} phoPQ ΔasdA18::TT araC |
| | P_{BAD} c2 |
| χ9388 | Δalr-3 ΔdadB4 ΔP_{phoPQ107}::TT araC P_{BAD} phoPQ ΔasdA21::TT araC |
| | P_{BAD} c2 |
| χ9389 | Δalr-3 ΔdadB4 ΔphoP233 ΔasdA21::TT araC P_{BAD} c2 |
| χ9412 | Δpmi-2426 Δ(gmd-fcl)-26 ΔP_{fur81}::TT araC P_{BAD} fur ΔP_{crp527}::TT araC |
| | P_{BAD} crp ΔasdA21::TT araC P_{BAD} c2 ΔaraE25 ΔaraBAD23 |
| | ΔrelA198::araC P_{BAD} lacI TT ΔP_{murA7}::TT araC P_{BAD} murA |
| χ9413 | Δpmi-2426 Δ(gmd-fcl)-26 ΔP_{phoPQ107}::TT araC P_{BAD} phoPQ |
| | ΔP_{crp527}::TT araC P_{BAD} crp ΔasdA18::TTaraC P_{BAD} c2 |
| | ΔaraE25 ΔaraBAD23 ΔrelA198::araC P_{BAD} lacI TT |
| | ΔP_{murA7}::TT araC P_{BAD} murA |
| χ9420 | Δpmi-2426 Δ(gmd-fcl)-26 ΔP_{phoPQ107}::TT araC P_{BAD} phoPQ |
| | ΔP_{crp527}::TT araC P_{BAD} crp ΔasdA18::TT araC P_{BAD} c2 |
| | ΔaraE25 ΔaraBAD23 ΔrelA198::araC P_{BAD} lacI TT |
| | ΔP_{murA7}::TT araC P_{BAD} murA ΔsifA26 |
| χ9442 | ΔP_{murA12}::TT araC P_{BAD} murA |
| χ9477 | ΔasdA27::TT araC P_{BAD} c2 |
| χ9514 | Δpmi-2426 Δ(gmd-fcl)-26 ΔP_{phoPQ107}::TT araC P_{BAD} phoPQ |
| | ΔP_{crp527}::TT araC P_{BAD} crp ΔaraE25 ΔaraBAD23 |
| | ΔasdA18::TT araC P_{BAD} c2 ΔrelA198::araC P_{BAD} lacI TT |
| | ΔP_{murA7}::TT araC P_{BAD} murA ΔsifA26 ΔrecF126 |
| χ9520 | ΔP_{murA12}::TT araC P_{BAD} murA ΔaraBAD23 |
| χ9521 | ΔP_{murA12}::TT araC P_{BAD} murA Δ(araC P_{BAD})-5::P22 P_{R} araBAD |
| χ9522 | ΔP_{murA7}::TT araC P_{BAD} murA ΔaraBAD23 |
| χ9523 | ΔasdA27::TT araC P_{BAD} c2 ΔaraBAD23 |
| χ9691 | ΔP_{murA12}::TT araC P_{BAD} murA ΔasdA27::TT araC P_{BAD} c2 |
| χ9692 | ΔP_{murA12}::TT araC P_{BAD} murA ΔasdA27::TT araC P_{BAD} c2 ΔaraBAD23 |
| χ9692 | ΔP_{murA12}::TT araC P_{BAD} murA ΔasdA27::TT araC P_{BAD} c2 Δ(araC |
| | P_{BAD})-5::P22 P_{R} araBAD |
| χ9744 | ΔP_{murA12}::TT araC P_{BAD} murA ΔasdA27::TT araC P_{BAD} c2 Δ(araC |
| | P_{BAD})-5::P22 P_{R} araBAD Δ(gmd-fcl)-26 |
| χ9745 | ΔP_{murA12}::TT araC P_{BAD} murA ΔasdA27::TT araC P_{BAD} c2 Δ(araC |
| | P_{BAD})-5::P22 P_{R} araBAD Δ(gmd-fcl)-26 Δpmi-2426 |
| χ9759 | Δpmi-2426 Δ(gmd-fcl)-26 ΔP_{fur81}::TT araC P_{BAD} fur |
| | ΔP_{crp527}::TT araC P_{BAD} crp ΔasdA27::TT araC P_{BAD} c2 ΔaraE25 |
| | ΔaraBAD23 ΔrelA198::araC P_{BAD} lacI TT ΔsopB1925 Δalr-3 ΔdadB4 |
| | ΔrecJ1315 |
| χ9760 | Δpmi-2426 Δ(gmd-fcl)-26 ΔP_{fur81}::TT araC P_{BAD} fur |
| | ΔP_{crp527}::TT araC P_{BAD} crp ΔasdA27::TT araC P_{BAD} c2 ΔaraE25 |
| | ΔaraBAD23 ΔrelA198::araC P_{BAD} lacI TT ΔsopB1925 Δalr-3 ΔdadB4 |
| | ΔrecF126 |
| χ9821 | ΔmurI-861 |
| χ9822 | Δpmi-2426 Δ(gmd-fcl)-26 ΔP_{fur81}::TT araC P_{BAD} fur |
| | ΔP_{crp527}::TT araC P_{BAD} crp ΔasdA27::TT araC P_{BAD} c2 |
| | ΔaraE25 ΔaraBAD23 ΔrelA198::araC P_{BAD} lacI TT |
| | ΔP_{murA7}::TT araC P_{BAD} murA |
| χ9830 | Δpmi-2426 Δ(gmd-fcl)-26 ΔP_{fur81}::TT araC P_{BAD} fur |
| | ΔP_{crp527}::TT araC P_{BAD} crp ΔasdA27::TT araC P_{BAD} c2 ΔaraE25 |
| | ΔaraBAD23 ΔrelA198::araC P_{BAD} lacI TT ΔsopB1925 Δalr-3 ΔdadB4 |
| | ΔrecF126 ΔrecJ1315 |
| χ9833 | ΔrecA62 |
| χ9834 | Δalr-3 ΔdadB4 ΔasdA33 ΔrecA62 |

### I. Recombinant Bacterium

As described above, in one embodiment, the invention encompasses a recombinant bacterium comprising at least one chromosomally encoded essential nucleic acid sequence that is altered so that it is not expressed, and at least one extrachromosomal vector. Each is described in more detail below.

(a) Chromosomally Encoded Essential Nucleic Acid that is Altered so that it is not Expressed

A recombinant bacterium of the invention comprises at least one chromosomally encoded essential nucleic acid sequence, wherein the essential nucleic acid sequence is altered so that it is not expressed. As described above, an essential nucleic acid is a native nucleic acid whose expression is necessary for cell viability or a metabolic activity essential for virulence. In some embodiments, an individual nucleic acid sequence is not essential, but the combination of one or more sequences, together, is essential. Stated another way, if the nucleic acid sequences in an essential combination are altered, so that they are not expressed, the cell is non-viable and/or avirulent.

A nucleic acid sequence that encodes a protein necessary for the formation of the peptidoglycan layer of the cell wall may be an essential nucleic acid. In one embodiment, an essential nucleic acid encodes a protein involved in D-alanine synthesis. For example, an essential nucleic acid may encode one or more alanine racemase proteins. In another embodiment, an essential nucleic acid may encode a protein involved in D-glutamate synthesis. In yet another embodiment, an essential nucleic acid may encode a protein involved in muramic acid synthesis. Such nucleic acid sequences are known in the art, and non-limiting examples may include asd, murA, murl, dap, alr, and dadB. In an alternative embodiment, a nucleic acid sequence that encodes a protein whose metabolic activity is essential for virulence may be an essential nucleic acid. Such nucleic acid sequences are also known in the art, and non-limiting examples may include aroA, aroC, aroD, aroE, ilvB, ilvC, ilvD or ilvE. [ANY OTHERS THAT SHOULD BE ADDED HERE?]

A recombinant bacterium of the invention may comprise more than one chromosomally encoded essential nucleic acid sequence that is altered so that it is not expressed. For instance, a recombinant bacterium may comprise two, three, four, five, or more than five different chromosomally encoded altered essential nucleic acid sequences.

Methods of making a recombinant bacterium comprising a chromosomally encoded essential nucleic acid sequence that is altered so that it is not expressed are known in the art and detailed in the examples. Non-limiting examples of suitable alterations are detailed below.

i. Essential Nucleic Acid Encoding a Protein Involved in D-Alanine Synthesis

In one embodiment, an essential nucleic acid may encode a protein involved in D-alanine synthesis, since D-alanine is a required constituent of the peptidoglycan layer of a bacterial cell wall. Gram-positive bacteria comprise only one alanine racemase, an enzyme necessary for D-alanine synthesis. Consequently, if the essential nucleic acid sequence encoding the Gram-positive alanine racemase is altered so that it is not expressed, the bacterium is non-viable. Gram-negative bacteria, however, comprise two alanine racemases. Consequently, it is the combination of both sequences that is essential, and the nucleic acid sequences encoding both alanine racemases need to be altered so that both sequences are not expressed. Suitable alterations may include deletion of the nucleic acid sequence encoding an alanine racemase. For instance, the combination of the deletions ΔaIr and ΔdadB will alter the essential combination such that neither racemase is expressed. Advantageously, an extrachromosomal vector need only encode one racemase to restore viability and/or virulence to the Gram-negative bacterium.

ii. Essential Nucleic Acid Encoding a Protein Involved in Muramic Acid Synthesis

In another embodiment, an essential nucleic acid may encode a protein involved in muramic acid synthesis, as muramic acid is another required constituent of the peptidoglycan layer of the bacterial cell wall. For example, an essential nucleic acid may be murA. It is not possible to alter murA by deletion, however, because a ΔmurA mutation is lethal and can not be isolated. This is because the missing nutrient required for viability is a phosphorylated muramic acid that cannot be exogenously supplied because enteric bacteria cannot internalize it. Consequently, the murA nucleic acid sequence may be altered to make expression of murA dependent on a nutrient (e.g., arabinose) that can be supplied during the growth of the bacterium. For example, the alteration may comprise a ΔP_{murA}::TT araC P_{BAD} murA deletion-insertion mutation. During in vitro growth of the bacterium, this type of mutation makes synthesis of muramic acid dependent on the presence of arabinose in the growth medium. During growth of the bacterium in a host, however, arabinose is absent. Consequently, the bacterium is non-viable and/or avirulent in a host unless the bacterium further comprises at least one extrachromosomal vector comprising a nucleic acid sequence, that when expressed, substantially functions as murA. Recombinant bacteria with a ΔP_{murA}::TT araC P_{BAD} murA deletion-insertion mutation grown in the presence of arabinose exhibit effective colonization of effector lymphoid tissues after oral vaccination prior to cell death due to cell wall-less lysing.

iii. Essential Protein Involved in D-Glutamate Synthesis

In yet another embodiment, an essential nucleic acid may encode a glutamate racemase, an enzyme essential for the synthesis of D-glutamic acid, which is another required constituent of the peptidoglycan layer of the bacterial cell wall. An essential nucleic acid encoding a glutamate racemase may be altered by deletion. For instance, the mutation Δmurl alters the nucleic acid sequence so that it is not expressed.

iv. Essential Protein Involved In DAP Synthesis

In still another embodiment, an essential nucleic acid may encode a protein involved in the synthesis of diaminopimelic acid (DAP). Various nucleic acid sequences are involved in the eventual synthesis of DAP, including dapA, dapB, dapC, dapD, dapE, dapF, and asd. Methods of altering an essential nucleic acid encoding a protein involved in the synthesis of DAP are known in the art. For instance, one of skill in the art may use the teachings of U.S. Pat. No. 6,872,547, hereby incorporated by reference in its entirety, for alterations that abolish DAP synthesis. In one example, the essential nucleic acid asdA may be altered by a ΔasdA mutation, so that asdA is not expressed. This eliminates the bacterium's ability to produce β-aspartate semialdehyde dehydrogenase, an enzyme essential for the synthesis of DAP.

v. More Than One Chromosomally Encoded Essential Nucleic Acid that is Altered

In exemplary embodiments of the invention, a recombinant bacterium may comprise more than one chromosomally encoded essential nucleic acid sequence that is altered so that it is not expressed and at least one extrachromosomal vector.

For instance, in one embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a first extrachromosomal vector, the vector comprising a nucleic acid comprising a nucleic acid sequence, that when expressed, substantially functions as the first essential nucleic acid sequence, and a second extrachromosomal vector, the vector comprising a nucleic acid sequence, that when expressed, substantially functions as the second essential nucleic acid sequence.

In another embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a third chromosomally encoded essential nucleic acid that is altered so that the third essential nucleic acid is not expressed, a first extrachromosomal vector, the vector comprising a nucleic acid comprising a nucleic acid sequence, that when expressed, substantially functions as the first essential nucleic acid sequence, a second extrachromosomal vector, the vector comprising a nucleic acid sequence, that when expressed, substantially functions as the second essential nucleic acid sequence, and a third extrachromosomal vector, the vector comprising a nucleic acid sequence, that when expressed, substantially functions as the third essential nucleic acid sequence.

In yet another embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a third chromosomally encoded essential nucleic acid that is altered so that the third essential nucleic acid is not expressed, a fourth chromosomally encoded essential nucleic acid that is altered so that the fourth essential nucleic acid is not expressed, a first extrachromosomal vector, the vector comprising a nucleic acid comprising a nucleic acid sequence, that when expressed, substantially functions as the first essential nucleic acid sequence, a second extrachromosomal vector, the vector comprising a nucleic acid sequence, that when expressed, substantially functions as the second essential nucleic acid sequence, a third extrachromosomal vector, the vector comprising a nucleic acid sequence, that when expressed, substantially functions as the third essential nucleic acid sequence, and a fourth extrachromosomal vector, the vector comprising a nucleic acid sequence, that when expressed, substantially functions as the fourth essential nucleic acid sequence.

In other embodiments, a recombinant bacterium may comprise more than four chromosomally encoded essential nucleic acid sequences that are each altered so that they are not expressed, and more than four corresponding extrachromosomal vectors. In each of the above embodiments, the extrachromosomal vectors may further comprise a nucleic acid sequence encoding one or more antigens, as detailed below.

By way of non-limiting example, suitable alterations in essential nucleic acid sequences may include an alteration selected from the group consisting of ΔasdA, any Δdap mutation, a ΔdadB mutation with a Δalr mutation, a ΔP_{murA}::TT araC P_{BAD} murA deletion-insertion mutation, a Δmurl mutation, a ΔaroA mutation, a ΔaroC mutation, a ΔaroD mutation, a ΔilvC mutation, and a ΔilvE mutation. For instance, a bacterium may comprise two, three, four, five, or more than five alterations in an essential nucleic acid sequence selected from the group consisting of ΔasdA, any Δdap mutation, a ΔdadB mutation with a Δalr mutation, a P_{murA}::TT araC P_{BAD} murA deletion-insertion mutation, a Δmurl mutation, a ΔaroA mutation, a ΔaroC mutation, a ΔaroD mutation, a ΔilvC mutation, and a ΔilvE mutation.

### (b) Extrachromosomal Vector

A recombinant bacterium of the invention also comprises an extrachromosomal vector. The vector comprises a nucleic acid sequence that when expressed, substantially functions as the chromosomally encoded essential nucleic acid that is not expressed. Furthermore, the vector typically also comprises a nucleic acid sequence that encodes at least on antigen of interest. As used herein, “vector” refers to an autonomously replicating nucleic acid unit. The present invention may be practiced with any known type of vector, including viral, cosmid, phasmid, and plasmid vectors. The most preferred type of vector is a plasmid vector. The term “extrachromosomal,” as used herein, refers to the fact that the vector is not contained within the bacterium's chromosomal DNA. The vector may comprise some sequences that are identical or similar to chromosomal sequences of the bacterium, however, the vectors used herein do not integrate with chromosomal sequences of the bacterium.

As is well known in the art, plasmids and other vectors may possess a wide array of promoters, multiple cloning sequences, transcription terminators, etc., and vectors may vary in copy number per bacterium. Selection of a vector may depend, in part, on the desired level of expression of the nucleic acid sequence substantially functioning as the essential nucleic acid. Additionally, the selection of a vector may depend, in part, on the level of expression of the nucleic acid sequence encoding an antigen of interest necessary to elicit an immune response.

For instance, in embodiments where the vector might encode a surface localized adhesin as the antigen, or an antigen capable of stimulating T-cell immunity, it may be preferable to use a vector with a low copy number such as at least two, three, four, five, six, seven, eight, nine, or ten copies per bacterial cell. A non-limiting example of a low copy number vector may be a vector comprising the pSC101 ori. In other cases, an intermediate copy number vector may be optimal for inducing desired immune responses. For instance, an intermediate copy number vector may have at least 10, 11, 12, 13, 14, 15, 16, 17, 18, 19, 20, 21, 22, 23, 24, 25, 26, 27, 28, 29, or 30 copies per bacterial cell. A non-limiting example of an intermediate copy number vector may be a vector comprising the p15A ori. In still other cases, a high copy number vector may be optimal for the induction of maximal antibody responses. A high copy number vector may have at least 31, 35, 40, 45, 50, 55, 60, 65, 70, 75, 80, 85, 90, 95, or 100 copies per bacterial cell. In some embodiments, a high copy number vector may have at least 100, 125, 150, 175, 200, 225, 250, 275, 300, 325, 350, 375, or 400 copies per bacterial cell. Non-limiting examples of high copy number vectors may include a vector comprising the pBR on or the pUC ori.

Additionally, vector copy number may be increased by selecting for mutations that increase plasmid copy number. These mutations may occur in the bacterial chromosome but are more likely to occur in the vector.

Vectors of the invention generally possess a multiple cloning site for insertion of a nucleic acid sequence that may be operably-linked to the promoter sequence and generally posses a transcription terminator (TT) sequence after a coding region. Preferably, vectors used herein do not comprise antibiotic resistance markers to select for maintenance of the vector.

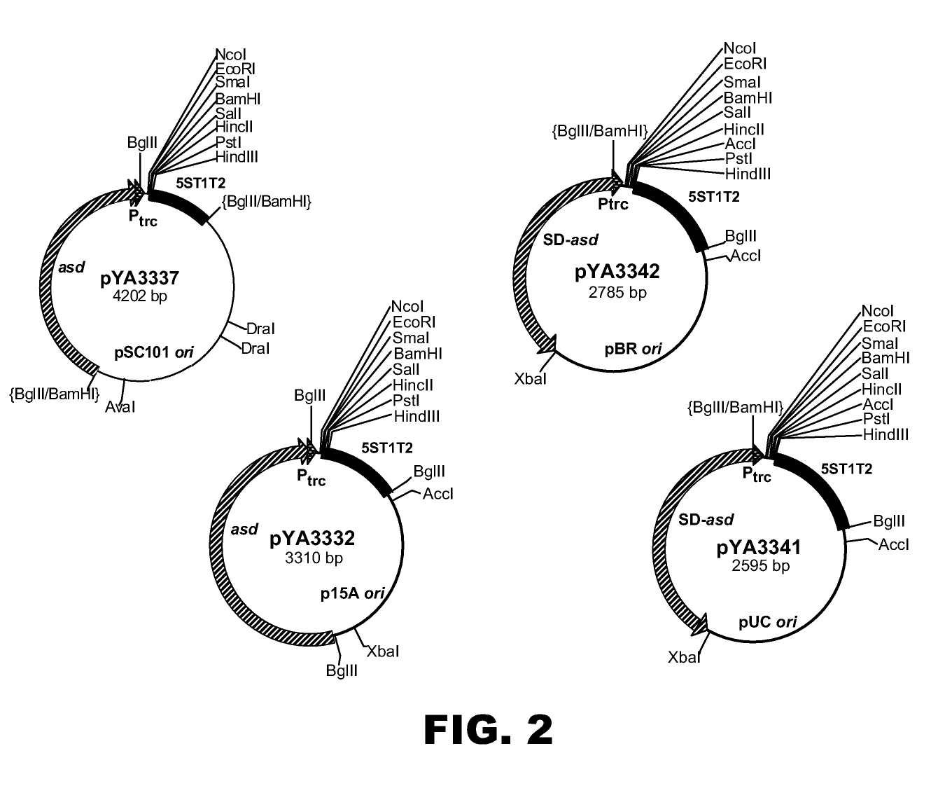

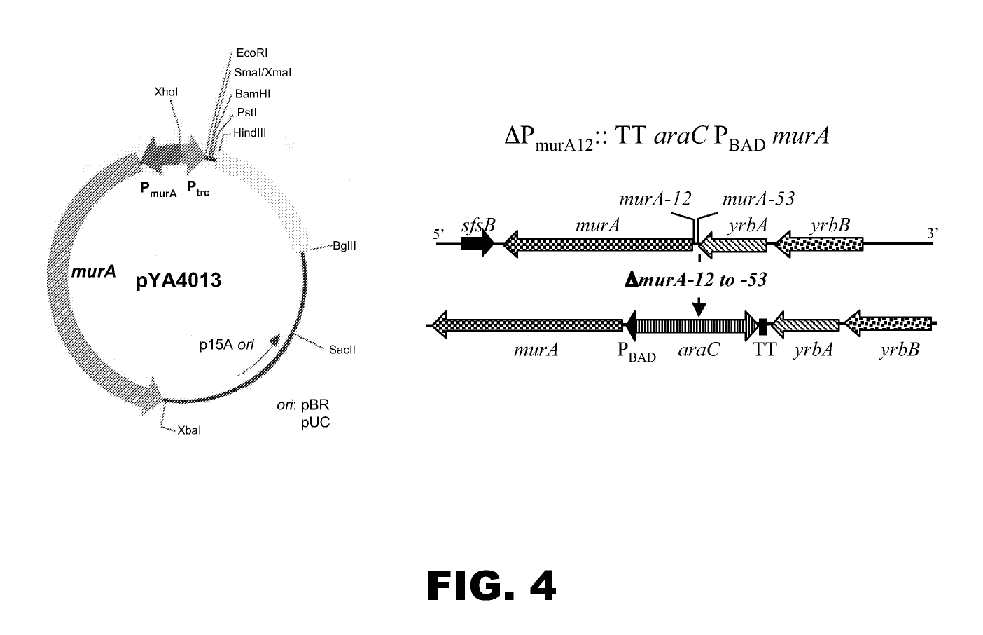

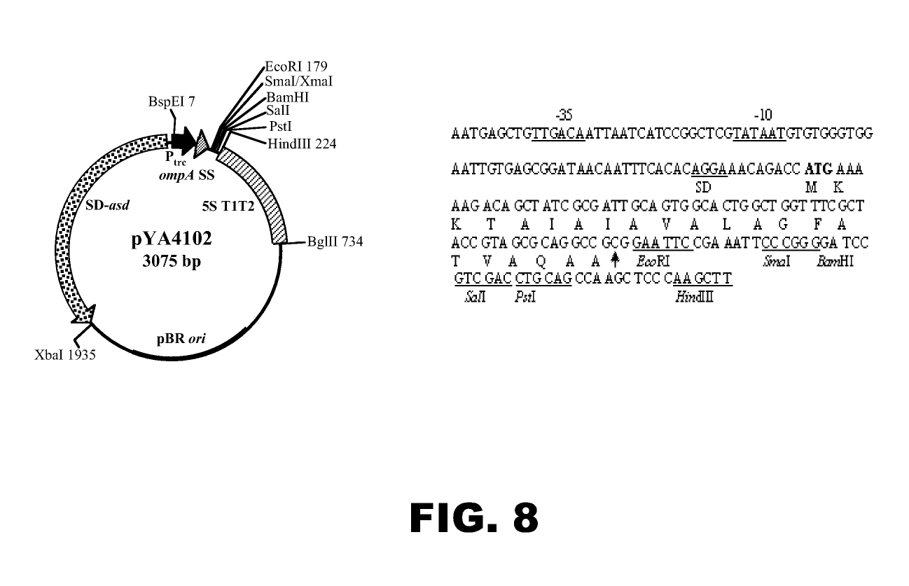

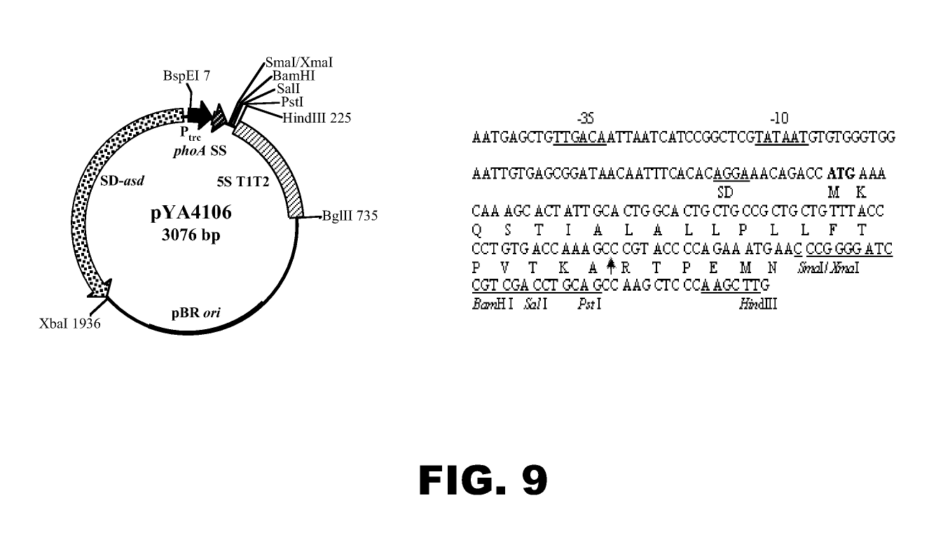

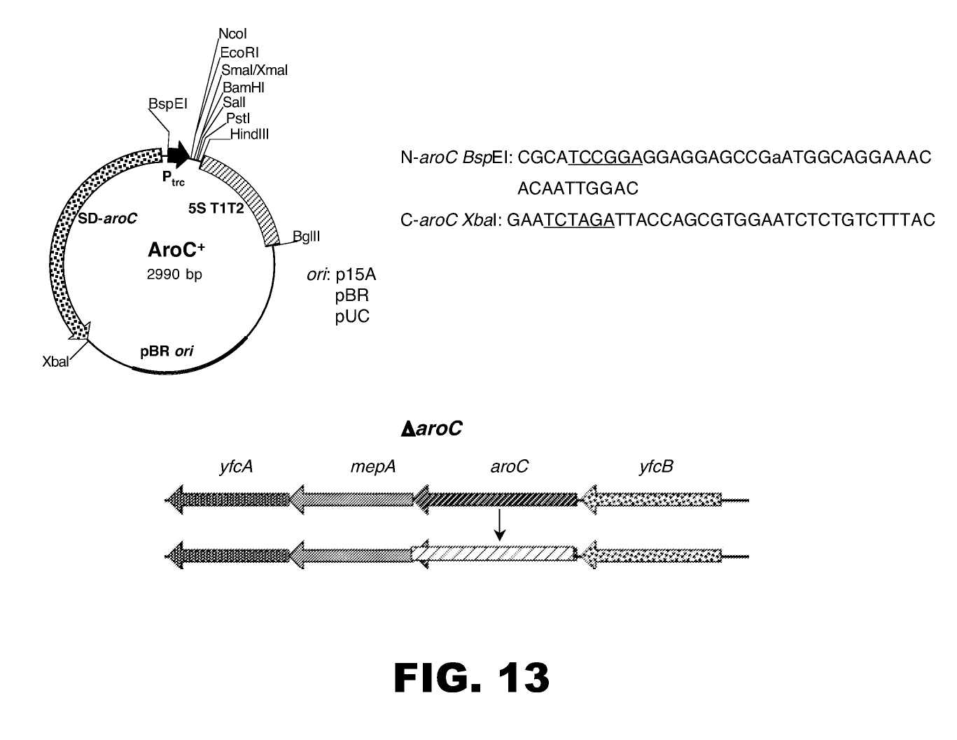

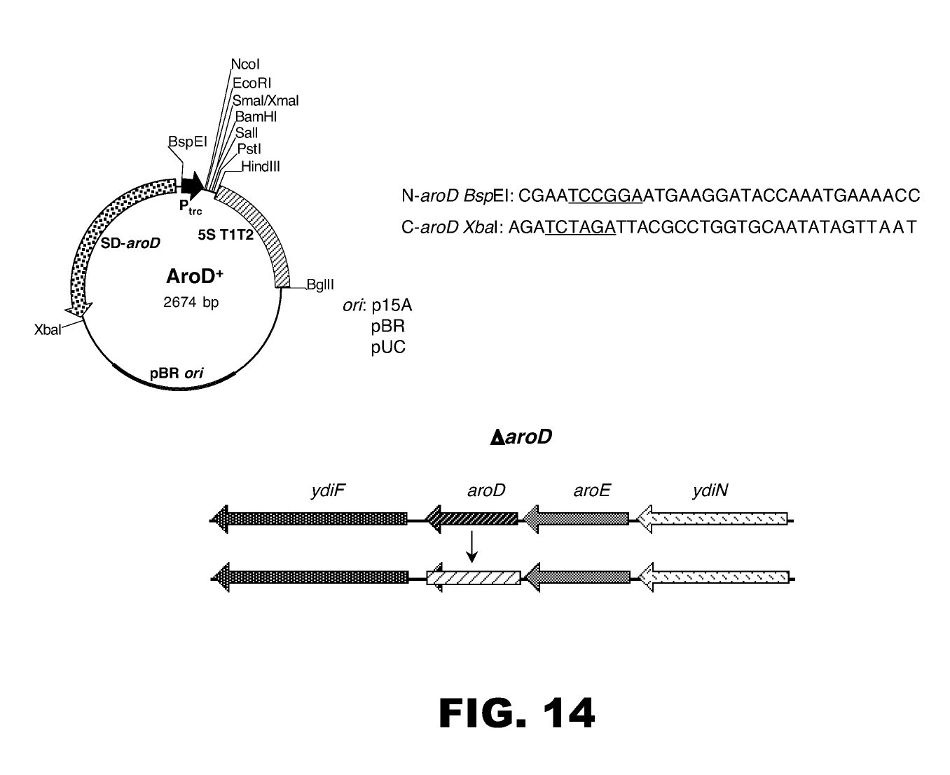

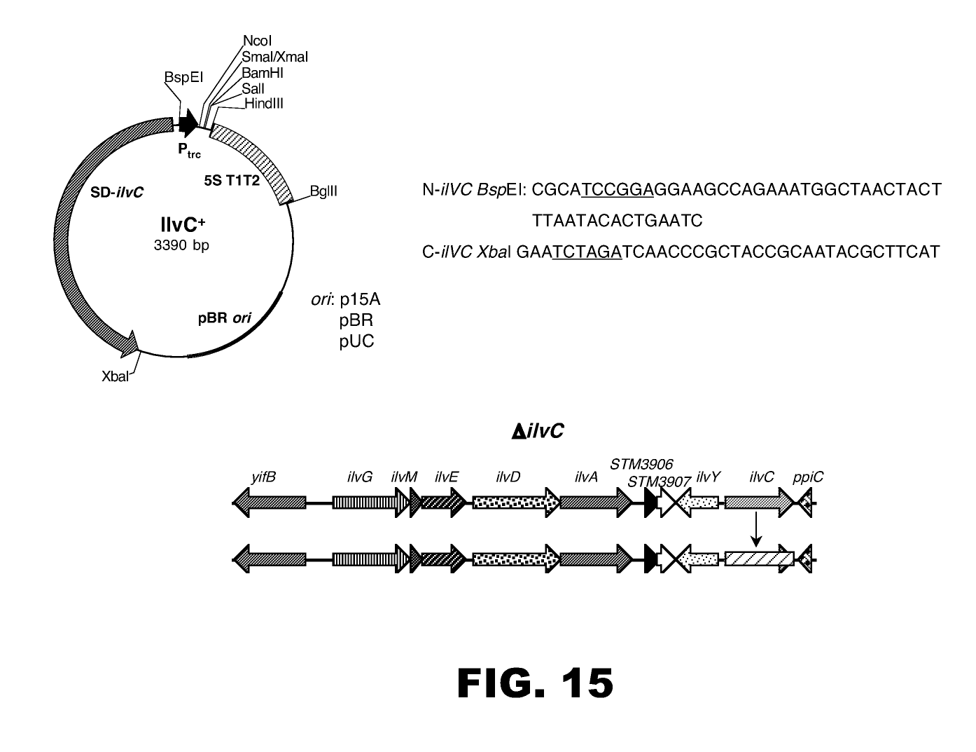

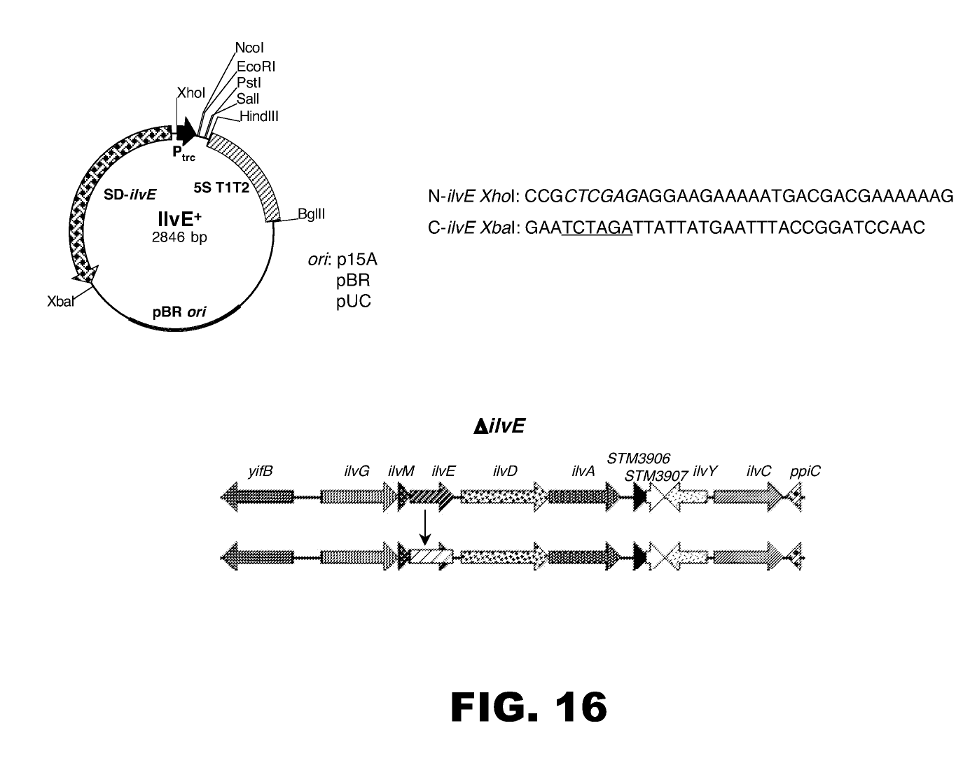

Examples of vectors that may be used are shown in FIGS. 2-9, 12-16 and in Table 2. These vectors comprise unique sequences to limit the recombination events between vectors. In addition, the vectors may comprise multiple cloning sites for inserting nucleic acid coding sequences for one or more antigen(s) of interest.

TABLE 2

| Plasmid | Properties |
|---|---|
| pYA3332 | p15A ori Asd⁺, 3425 bp |
| pYA3337 | pSC101 ori Asd⁺, 4343 bp |
| pYA3341 | pUC ori Asd⁺, 2771 bp |
| pYA3342 | pBR ori Asd⁺, 3012 bp |
| pYA3493 | pBR ori Asd⁺ bla SS, 3113 bp, |
| pYA3620 | pBR ori Asd⁺, bla SS bla CT, 3169 bp |
| pYA3634 | pBR ori bla SS, Asd⁺ PspA_{Rx1} aa 3-257 |
| pYA3681 | pBR ori araC P_{BAD} SD-GTG asdA SD-GTG murA P22 P_{R} anti-sense mRNA |
| pYA3703 | pBR ori araC P_{BAD} asd P_{trc} sopE::ESAT-6-HA, 4904 bp |
| pYA3712 | pBR ori pYA3681 harboring bla SS PspA_{Rx1} aa 3-257(codon optimized) |
| pYA3725 | pBR ori araC P_{BAD} asd P_{trc} sopE (Nt)::CFP-10::ESAT-6, 5384 bp |
| pYA3744 | pBR ori Asd⁺ P_{trc} phoA SS PspC aa 4-404, 4285 bp |
| pYA3747 | pBR ori Asd⁺ Pₗₚₚ ompA SS PspA_{Rx1} aa 3-285 |
| pYA3791 | pUC ori Asd⁺ P_{trc} HBV core aa 1-78 preS1 aa 20-47 |
| | HBV core aa 79-144 preS2 aa 120-151 HBV core aa 145-147, |
| | HBV core aa 179-183, 3472 bp |
| pYA3792 | pBR ori Asd⁺ P_{trc} HBV core aa 1-78 preS1 aa 20-47 |
| | HBV core aa 79-144 preS2 aa 120-151 HBV core aa 145-147, |
| | HBV core aa 179-183, 3662 bp |
| pYA3793 | pUC ori Asd⁺ P_{trc} HBV core aa 1-77 preS1 aa 20-47 |
| | HBV core aa 84-144 preS2 aa 120-151 HBV core aa 145-147, |
| | HBV core aa 179-183, 4599 bp |
| pYA3794 | pBR ori Asd⁺ P_{trc} HBV core aa1-77 preS1 aa 20-47 |
| | HBV core aa 84-144 preS2 aa 120-151 HBV core aa 145-147, |
| | HBV core aa 179-183, 3454 bp |
| pYA3795 | pUC ori Asd⁺ P_{trc} HBV core aa 1-74 preS1 aa 20-47 |
| | HBV core aa 87-144 preS2 aa 120-151 HBV core aa 145-147, |
| | HBV core aa 179-183, 3644 bp |
| pYA3796 | pBR ori Asd⁺ P_{trc} HBV core aa 1-74 preS1 aa 20-47 |
| | HBV core aa 87-144 preS2 aa 120-151 HBV core aa 145-147, |
| | HBV core aa179-183, 4581 bp |
| pYA3797 | pUC ori Asd⁺ P_{trc} HBV core aa1-69 preS1 aa 20-47 |
| | HBV core aa 91-144 preS2 aa 120-151 HBV core aa 145-147, |
| | HBV core aa 179-183, 3436 bp |
| pYA3744 | pBR ori phoA SS Asd⁺ P_{trc} PspC aa 4-404, 4285 bp |
| pYA3747 | pBR ori ompA SS Pₗₚₚ Asd⁺ PspA_{Rx1} aa 3-285 |
| pYA3791 | pUC ori Asd⁺ P_{trc} HBV core aa 1-78 preS1 aa 20-47 |
| | HBV core aa 79-144 preS2 aa 120-151 HBV core aa 145-147, |
| | HBV core aa 179-183, 3472 bp |
| pYA3792 | pBR ori Asd⁺ P_{trc} HBV core aa 1-78 preS1 aa 20-47 |
| | HBV core aa 79-144 preS2 aa 120-151 HBV core aa 145-147, |
| | HBV core aa 179-183, 3662 bp |
| pYA3793 | pUC ori Asd⁺ P_{trc} HBV core aa 1-77 preS1 aa 20-47 |
| | HBV core aa 84-144 preS2 aa 120-151 HBV core aa 145-147, |
| | HBV core aa 179-183, 4599 bp |
| pYA3794 | pBR ori Asd⁺ P_{trc} HBV core aa1-77 preS1 aa 20-47 |
| | HBV core aa 84-144 preS2 aa 120-151 HBV core aa 145-147, |
| | HBV core aa 179-183, 3454 bp |
| pYA3795 | pUC ori Asd⁺ P_{trc} HBV core aa 1-74 preS1 aa 20-47 |
| | HBV core aa 87-144 preS2 aa 120-151 HBV core aa 145-147, |
| | HBV core aa 179-183, 3644 bp |
| pYA3796 | pBR ori Asd⁺ P_{trc} HBV core aa 1-74 preS1 aa 20-47 |
| | HBV core aa 87-144 preS2 aa 120-151 HBV core aa 145-147, |
| | HBV core aa179-183, 4581 bp |
| pYA3797 | pUC ori Asd⁺ P_{trc} HBV core aa1-69 preS1 aa 20-47 |
| | HBV core aa 91-144 preS2 aa 120-151 HBV core aa 145-147, |
| | HBV core aa 179-183, 3436 bp |
| pYA3798 | pBR ori Asd⁺ P_{trc} HBV core aa 1-69 preS1 aa 20-47 |
| | HBV core aa 91-144 preS2 aa 120-151 HBV core aa 145-147, |
| | HBV core aa 179-183, 3626 bp |
| pYA3802 | pBR ori bla SS bla CT Asd⁺ P_{trc} PspA_{Rx1} aa 3-285, 3995 bp |
| pYA3833 | pBR ori DadB⁺ P22P_{L} phoP, 3.7 kb |
| pYA3861 | p15A ori DadB⁺ P22P_{L} phoP, 3.4 kb |
| pYA3862 | pUC ori DadB⁺ P22P_{L} phoP, 3.5 kb |
| pYA3869 | pSC101 ori Asd⁺ SopE⁺, 4399 bp |
| pYA3870 | p15A ori Asd⁺ SopE⁺, 3481 bp |
| pYA3920 | p15A ori P_{asd} asd P_{sopE} sopE (Nt)::ESAT-6, 3758 bp |
| pYA3940 | pBR ori bla SS::Ag85A asd, 3989 bp |
| pYA3941 | pBR ori bla SS::Ag85A::bla CT asd, 4037 bp |
| pYA3950 | p15A ori P_{asd} asd P_{sopE} sopE::CFP-10::ESAT-6, 4036 bp |
| pYA3911 | p15A ori DadB⁺ Pₗₚₚ ompA SS, 3.4 kb |
| pYA4012 | p15A ori MurA⁺ P_{phoA} phoA SS, 3107 bp |
| pYA4013 | p15A ori MurA⁺ P_{trc}, 2984 bp |
| pYA4014 | p15A ori DadB⁺ P22P_{L}, 2743 bp |
| pYA4015 | pBR ori DadB⁺ P22P_{L}, 3017 bp |
| pYA4016 | pUC ori DadB⁺ P22P_{L}, 2796 bp |
| pYA4019 | pSC101 ori Asd⁺ P_{trc}, pagL, 5.0 kb |
| pYA4020 | pBR ori DadB⁺ P22P_{L}, lpxE, 3.6 kb |
| pYA4021 | p15A ori DadB⁺ P22P_{L}, lpxE, 3.4 kb |
| pYA4022 | pUC ori DadB⁺ P22P_{L}, lpxE, 3.4 kb |
| pYA4028 | pBR ori Asd⁺ P_{trc} bla SS PspC aa 4-404, 4322 bp |
| pYA4029 | pBR ori Asd⁺ P_{trc} bla SS PspC aa 4-445 with the proline rich domain, 4452 bp |
| pYA4088 | pBR ori Asd⁺ P_{trc} bla SS PspA_{Rx1} aa 3-285, 3927 bp |
| pYA4099 | p15A ori DadB⁺ Pₗₚₚ ompA SS PspC aa 4-404, 4309 bp |
| pYA4100 | pBR ori Asd⁺ Pₗₚₚ ompA SS PspC aa 4-404 |
| pYA4102 | pBR ori ompA SS Asd⁺, 3075 bp |
| pYA4106 | pBR ori Asd⁺ phoA SS, 3076 bp |
| pYA4116 | pUC ori SD-improved DadB⁺ P22 P_{L}, 2757 bp |
| pYA4187 | p15A ori DadB⁺ Pₗₚₚ ompA SS ESAT-6::3xFLAG, 3629 bp |
| pYA4202 | pBR ori Asd⁺ P_{trc} ompA SS PspC aa 4-404, 4284 bp |
| pYA4205 | p15A ori DadB⁺ Pₗₚₚ ompA SS PspA_{Rx1} aa 3-257, 3883 bp |
| pYA4240 | pBR ori P_{murA} MurA⁺ P_{trc}, 3.2 kb |
| pYA4241 | pUC ori P_{murA} MurA⁺ P_{trc}, 3.0 kb |
| pYA4251 | p15A ori P_{asd} asd P_{sopE}sopE(Nt)::ESAT-6::ESAT-6::ESAT-6, 4322 bp |
| pYA4257 | p15A ori P_{asd} asd P_{sopE}sopE(Nt)::ESAT-6::ESAT-6::CFP-10, 4327 bp |
| pYA4266 | pBR ori Asd⁺ P_{trc} ompA SS PspA_{Rx1} aa 3-285, 3927 bp |
| pYA4267 | pBR ori Asd⁺ P_{trc} phoA SS PspA_{Rx1} aa 3-285, 3928 bp |
| pYA4269 | pBR ori Asd⁺ P_{trc} bla SS PspC aa 4-404 bla CT, 4378 bp |
| pYA4346 | p15A ori SD-improved DadB⁺ P22 P_{L}, 2704 bp |
| pYA4347 | pBR ori SD-improved DadB⁺ P22 P_{L}, 2978 bp |
| pYA4431 | pBR ori Asd⁺ P_{trc} bla SS::PspA_{Rx1} aa 3-285::PspA_{EF5668} aa 4-417::bla |
| | CT, 5243 bp |
| pYA4432 | pBR ori Asd⁺ P_{trc} bla SS::PspA_{EF5668} aa 4-417::PspA_{Rx1} aa 3-285, |
| | 5175 bp |
| pYA4433 | pBR ori Asd⁺ P_{trc} bla SS::PspA_{Rx1} aa 3-285::PspA_{EF5668} aa 4-417, |
| | 5175 bp |
| pYA4522 | p15A ori P_{murA} MurA⁺ P_{sifA} sscB::sseF::CFP-10::ESAT- |
| | 6::3xFLAG, 5193 bp |
| pYA4523 | p15A ori P_{murA} MurA⁺ P_{sifA} bla SS CFP-10::ESAT-6::3xFLAG, |
| | 4002 bp |
| pYA4526 | p15A ori P_{murA} MurA⁺ P_{sifA} dsbA SS CFP-10::ESAT-6::3xFLAG, |
| | 3992 bp |
| pYA4527 | p15A ori P_{murA} MurA⁺ P_{sifA} CFP-10::ESAT-6::3x FLAG, 3830 bp |
| pYA4552 | pUC ori WSDΩAAAC GTG-DadB⁺, 2.7 kb |
| pYA4554 | pBR ori WSDΩAAAC GTG-DadB⁺, 2.9 kb |
| pYA4557 | pUC ori SD (AGGA) MurA⁺ P_{trc}, 2.9 kb |
| pYA4558 | pUC ori SD (AGGA) GTG-MurA⁺ P_{trc}, 2.9 kb |
| pYA4596 | pBR ori P_{murA} SD (AGAA) GTG-MurA⁺ P_{trc}, 3.2 kb |
| pYA4597 | pBR ori P_{murA} WSD (AAGG) GTG-MurA⁺ P_{trc}, 3.2 kb |

p = plasmid
:: = insertion or fusion
CT = C-terminal
Ω = insertion
P = promoter
aa = amino acid
Nt = N-terminal
SD = Shine-Dalgarno sequence
SS = signal sequence
WSD = weaker SD sequence

i. Nucleic Acid that Substantially Functions as an Essential Nucleic Acid

An extrachromosomal vector of the invention comprises a nucleic acid, that when expressed, substantially functions as the essential nucleic acid that was chromosomally altered so that it is not expressed. The phrase “substantially functions,” as used herein, means that the expression of the nucleic acid sequence encoded by the vector restores viability and/or virulence to the recombinant bacterium comprising a chromosomally encoded essential nucleic acid sequence that was altered so that it was not expressed. The nucleic acid, that when expressed, substantially functions as the essential nucleic acid that was chromosomally altered, may, in some embodiments, be derived from the same strain of bacteria as the essential nucleic acid. In other embodiments, the nucleic acid, that when expressed, substantially functions as the essential nucleic acid that was chromosomally altered, may be derived from a different strain of bacteria as the essential nucleic acid.

As described above, if the chromosomally encoded essential nucleic acid that is not expressed encodes a protein such as Alr, DadB, Dap, MurA, Murl, Asd, AroA, AroC, AroD, IlvC, and IlvE, then the nucleic acid sequence encoded by the extrachromosomal vector will substantially function as a nucleic acid sequence encoding Alr, DadB, Dap, MurA, Murl, Asd, AroA, AroC, AroD, IlvC, and IlvE respectively.

An extrachromosomal vector of the invention vector may also comprise a promoter operably-linked to the nucleic acid sequence that substantially replaces the function of an essential nucleic acid sequence. This may depend, however, on the copy number of the vector. For instance, if the vector is a high copy number vector, the nucleic acid sequence that substantially replaces the function of an essential nucleic acid may not be operably-linked to a promoter but may instead only comprise a Shine-Dalgarno (SD) sequence. Alternatively, if the vector is a low copy number vector, the nucleic acid sequence that substantially replaces the function of an essential nucleic acid may be operably-linked to a promoter. Such a promoter may be a weak promoter, a strong promoter, a regulated promoter or a constitutive promoter, depending, in part, on the desired level of expression of the sequence that substantially replaces the function of an essential nucleic acid sequence. The “desired level,” as used herein, is at least the level necessary to render the bacterium viable and/or virulent.

In certain embodiments, the nucleic acid sequence encoded by the extrachromosomal vector may be modified to alter the level of transcription of the nucleic acid. For instance, such alterations may include modifying the SD sequence and or the sequence of the start codon.

ii. Nucleic Acid Sequence Encoding at Least One Antigen

As used herein, “antigen” refers to a biomolecule capable of eliciting an immune response in a host. In some embodiments, an antigen may be a protein, or fragment of a protein, or a nucleic acid. In an exemplary embodiment, the antigen elicits a protective immune response. As used herein, “protective” means that the immune response contributes to the lessening of any symptoms associated with infection of a host with the pathogen the antigen was derived from or designed to elicit a response against. For example, a protective antigen from a pathogen, such as Mycobacterium, will induce an immune response that helps to ameliorate symptoms associated with Mycobacterium infection or reduces the morbidity and mortality associated with infection with the pathogen. The use of the term “protective” in this invention does not necessarily require that the host is completely protected from the effects of the pathogen.

Antigens may be from bacterial, viral, mycotic and parasitic pathogens, and may be designed to protect against bacterial, viral, mycotic, and parasitic infections, respectively. Alternatively, antigens may be derived from gametes, provided they are gamete specific, and may be designed to block fertilization. In another alternative, antigens may be tumor antigens, and may be designed to decrease tumor growth. It is specifically contemplated that antigens from organisms newly identified or newly associated with a disease or pathogenic condition, or new or emerging pathogens of animals or humans, including those now known or identified in the future, may be expressed by a bacterium detailed herein. Furthermore, antigens for use in the invention are not limited to those from pathogenic organisms. The selection and recombinant expression of antigens has been previously described by Schodel (1992) and Curtiss (1990). Immunogenicity of the bacterium can be augmented and/or modulated by constructing strains that also express sequences for cytokines, adjuvants, and other immunomodulators.

Some examples of microorganisms useful as a source for antigen are listed below. These may include microoganisms for the control of plague caused by Yersinia pestis and other Yersinia species such as Y. pseudotuberculosis and Y. enterocolitica, of gonorrhea caused by Neisseria gonorrhoea, of syphilis caused by Treponema pallidum, and of venereal diseases as well as eye infections caused by Chlamydia trachomatis. Species of Streptococcus from both group A and group B, such as those species that cause sore throat or heart diseases, Erysipelothrix rhusiopathiae, Neisseria meningitidis, Mycoplasma pneumoniae and other Mycoplasma-species, Hemophilus influenza, Bordetella pertussis, Mycobacterium tuberculosis, Mycobacterium leprae, other Bordetella species, Escherichia coli, Streptococcus equi, Streptococcus pneumoniae, Brucella abortus, Pasteurella hemolytica and P. multocida, Vibrio cholera, Shigella species, Borrellia species, Bartonella species, Heliobacter pylori, Campylobacter species, Pseudomonas species, Moraxella species, Brucella species, Francisella species, Aeromonas species, Actinobacillus species, Clostridium species, Rickettsia species, Bacillus species, Coxiella species, Ehrlichia species, Listeria species, and Legionella pneumophila are additional examples of bacteria within the scope of this invention from which antigen nucleic acid sequences could be obtained. Viral antigens may also be used. Viral antigens may be used in antigen delivery microorganisms directed against viruses, either DNA or RNA viruses, for example from the classes Papovavirus, Adenovirus, Herpesvirus, Poxvirus, Parvovirus, Reovirus, Picornavirus, Myxovirus, Paramyxovirus, Flavivirus or Retrovirus. Antigens may also be derived from pathogenic fungi, protozoa and parasites.

Certain embodiments encompass an allergen as an antigen. Allergens are substances that cause allergic reactions in a host that is exposed to them. Allergic reactions, also known as Type I hypersensitivity or immediate hypersensitivity, are vertebrate immune responses characterized by IgE production in conjunction with certain cellular immune reactions. Many different materials may be allergens, such as animal dander and pollen, and the allergic reaction of individual hosts will vary for any particular allergen. It is possible to induce tolerance to an allergen in a host that normally shows an allergic response. The methods of inducing tolerance are well-known and generally comprise administering the allergen to the host in increasing dosages.

It is not necessary that the vector comprise the complete nucleic acid sequence of the antigen. It is only necessary that the antigen sequence used be capable of eliciting an immune response. The antigen may be one that was not found in that exact form in the parent organism. For example, a sequence coding for an antigen comprising 100 amino acid residues may be transferred in part into a recombinant bacterium so that a peptide comprising only 75, 65, 55, 45, 35, 25, 15, or even 10, amino acid residues is produced by the recombinant bacterium. Alternatively, if the amino acid sequence of a particular antigen or fragment thereof is known, it may be possible to chemically synthesize the nucleic acid fragment or analog thereof by means of automated nucleic acid sequence synthesizers, PCR, or the like and introduce said DNA sequence into the appropriate copy number vector.

In another alternative, a vector may comprise a long sequence of nucleic acid encoding several nucleic acid sequence products, one or all of which may be antigenic. In some embodiments, a vector of the invention may comprise a nucleic acid sequence encoding at least one antigen, at least two antigens, at least three antigens, at least four antigens, or more than four antigens. These antigens may be encoded by two or more open reading frames operably linked to be expressed coordinately as an operon. Alternatively, the two or more antigens may be encoded by a single open reading frame to generate synthesis of a fusion protein.

In certain embodiments, an antigen of the invention may comprise a B cell epitope or a T cell epitope. Alternatively, an antigen to which an immune response is desired may be expressed as a fusion to a carrier protein that contains a strong promiscuous T cell epitope and/or serves as an adjuvant and/or facilitates presentation of the antigen to enhance, in all cases, the immune response to the antigen or its component part. This can be accomplished by methods known in the art. Fusion to tenus toxin fragment C, CT-B, LT-B and hepatitis virus B core are particularly useful for these purposes, although other epitope presentation systems are well known in the art.

In further embodiments, an antigen of the invention may comprise a secretion signal, as described below. In other embodiments, an antigen of the invention may be toxic to the recombinant bacterium. In certain embodiments, the above nucleic acid sequences encoding an antigen may be placed under the control of a regulated promoter such that the nucleic acid sequence encoding the antigen is no expressed in vitro, but is expressed during growth of the bacterium in a host. In other embodiments, a vector of the invention may comprise a strong promoter for driving expression of the nucleic acid sequences encoding antigen(s) of interest. Examples of promoters that may be used include, but are not limited to P_{trc}, P_{L}, P_{R}, P_{Ipp}, P_{phoA} and the promoters shown in the figures. These promoters may contain operator sequences that recognize repressor proteins such as Lacl, C2, or C1 to enable regulation of the expression of nucleic acid sequences encoding the antigen(s) of interest.

In another embodiment, an extrachromosomal vector of the invention may be used to express polynucleotide sequences of various lengths. In one embodiment, the size of the polynucleotide sequence inserted into the vector is about 1 kb to about 10 kb, more preferably about 2 kb to about 7 kb, and even more preferably about 2 kb to about 5 kb. The biological principles governing for the bacterium's energy expenditure and the bacterium's ability to produce proteins efficiently for one versus multiple antigens are equally applicable here when considering how large of a sequence to include in the vector(s). In many cases, the nucleic acid sequences encode one or more antigens of interest. For instance, a nucleic acid sequence may encode a fusion of two or more antigens.

Another consideration that one of skill should consider is the copy number of the plasmid. The copy number of the plasmid should be inversely correlated to the amount of the insert such that the Salmonella does not express so much antigen that its fitness is compromised by exhaustion.

Antigens of interest may be protective antigens, which modulate an immune response in the individual. The modulation of an immune response may be in the form of the innate immune system, mucosal immune response, cellular immune response or humoral immune response. In a preferred embodiment, the immune response acts in a manner to target the antigen of interest, e.g., a pathogen such as Mycobacterium tuberculosis (Mtb) or Streptococcus pneumoniae, such that the pathogen is destroyed by the immune system. The immune response may also ameliorate the physical symptoms associated with infection with a pathogen or be stimulated to combat the infection more effectively.

In one embodiment, the antigens of interest include PspA and PspC from Streptococcus pneumoniae, In other embodiments, the antigens of interest is selected from any of the antigens listed in Table 3.

TABLE 3

| | |
|---|---|
| Antigens | Description |

Pneumococcal antigens

| | |
|---|---|
| PspA | Rx1 aa 3-285 (codon optimized) |
| | Rx1 aa 3-257 (original and codon optimized) |
| | EF5668 aa 79-308 (original and codon optimized) |
| | EF5668 aa 79-353 (original and codon optimized) |
| PspA Fusion | Rx1 aa 3-285::EF5668 aa 4-417 (codon optimized) |
| | EF5668 aa 4-417::Rx1 aa 3-285 (codon optimized) |
| PspC | L81905 aa 4-404 (original and codon optimized) |
| | L81905 aa 4-444 (original and codon optimized) |
| | EF6796 aa 3-587 (original and codon optimized) |
| | EF6796 aa 3-652 (codon optimized) |
| PsaA | aa 20-210 |
| | aa 21-210 |
| | aa 17-210 |
| | aa 17-210 without aa 20 |
| Ply | aa 1-471 D39 |

Mycobacterium tuberculosis

| | |
|---|---|
| ESAT-6 | aa 1-95 |
| CFP-10 | aa 1-100 |
| Ag85A | aa 1-338 |
| Mtb39A (PPE18) | aa 1-391 |

Yersinia pestis

| | |
|---|---|
| Vir (LcrV) | aa 131-327 (codon optimized) |
| Psn | aa 1-673 (entire protein) |
| Pla | aa 1-312 (entire protein) |
| PsaA (pH6 | aa 1-163 (codon optimized) |
| antigen) | |

Hepatitis B virus

| | |
|---|---|
| Pre-S1 | aa 20-47 |
| Pre-S2 | aa122-151 |

Influenza

| | |
|---|---|
| Hemagglutinin | aa 1-565 influenza A/WSN/33 virus (H1N1) |
| M2e | 23 aa |

aa: amino acid

iii. Antigen Delivery System

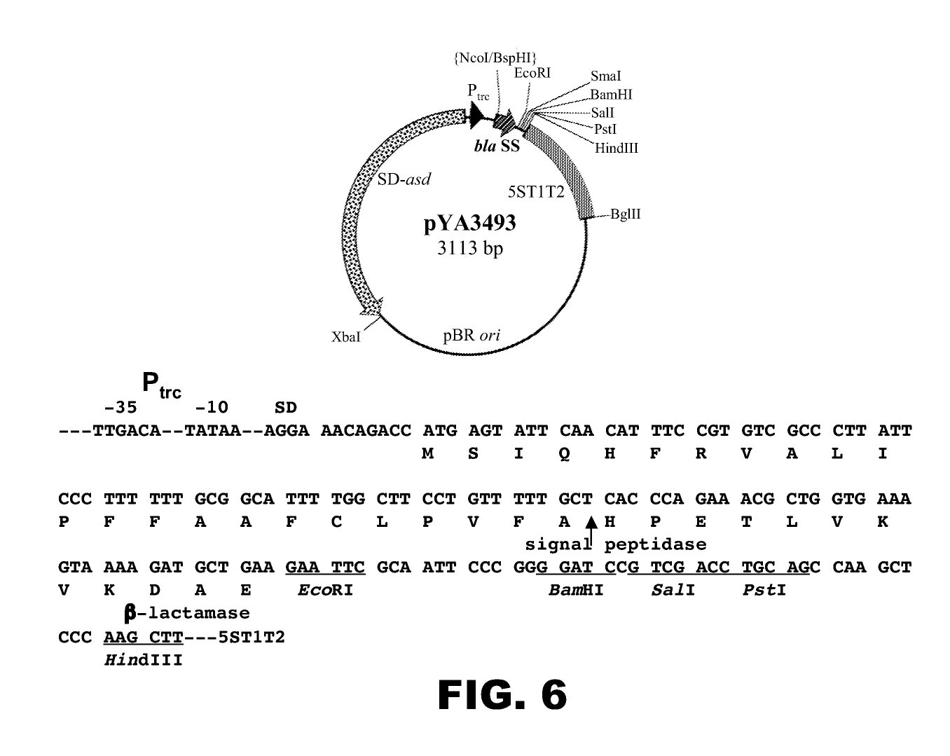

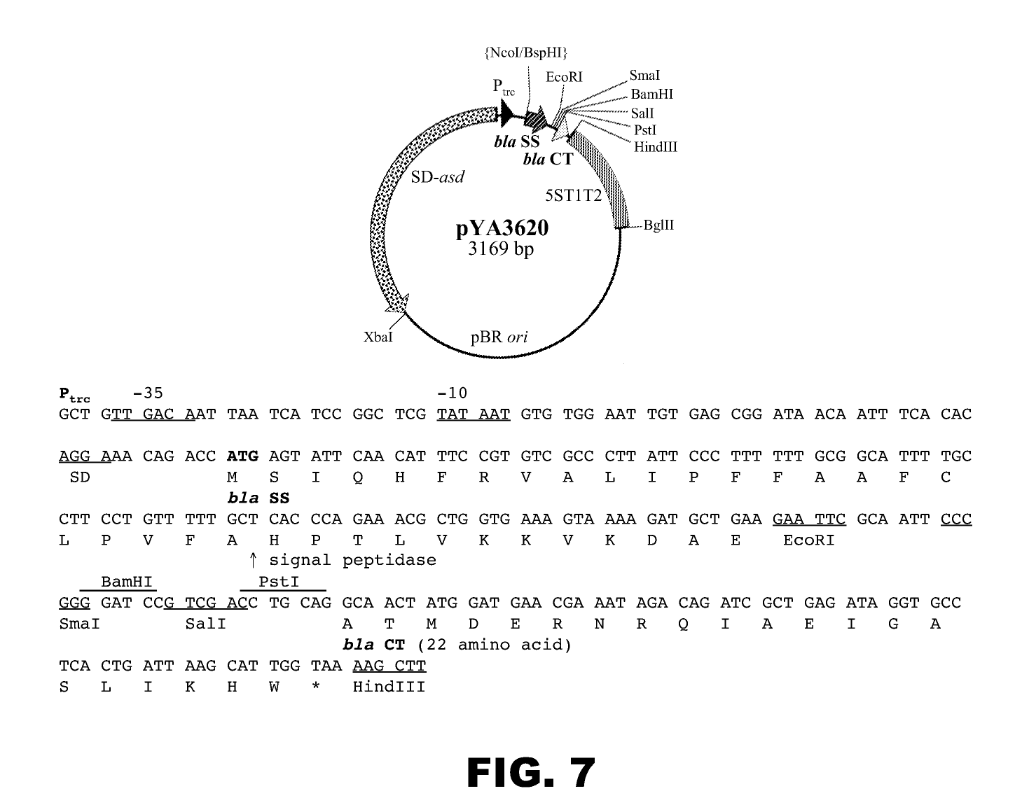

In addition, the vectors may be designed for various types of antigen delivery systems. The system that is selected will depend, in part, on the immune response desired. For example, if an antibody response is desired, then a Type II secretion system may be used. Examples of Type II secretion systems are well-known in the art, for instance, the β-lactamase secretion system may be used. FIGS. 6-9 illustrate examples of Type II secretion systems that may be used. The use of a Type II secretion system with the signal sequence located at the N-terminus (FIG. 6) is useful for secretion of many antigens while a Type II secretion system that combines a signal sequence located at the N-terminus with a segment of the C-terminus portion of β-lactamase (FIG. 7) often improves secretion of the antigen encoded by the nucleic acid sequence between the N-terminus segment and the C-terminus segment. This may in turn improve the immune response to the antigen.

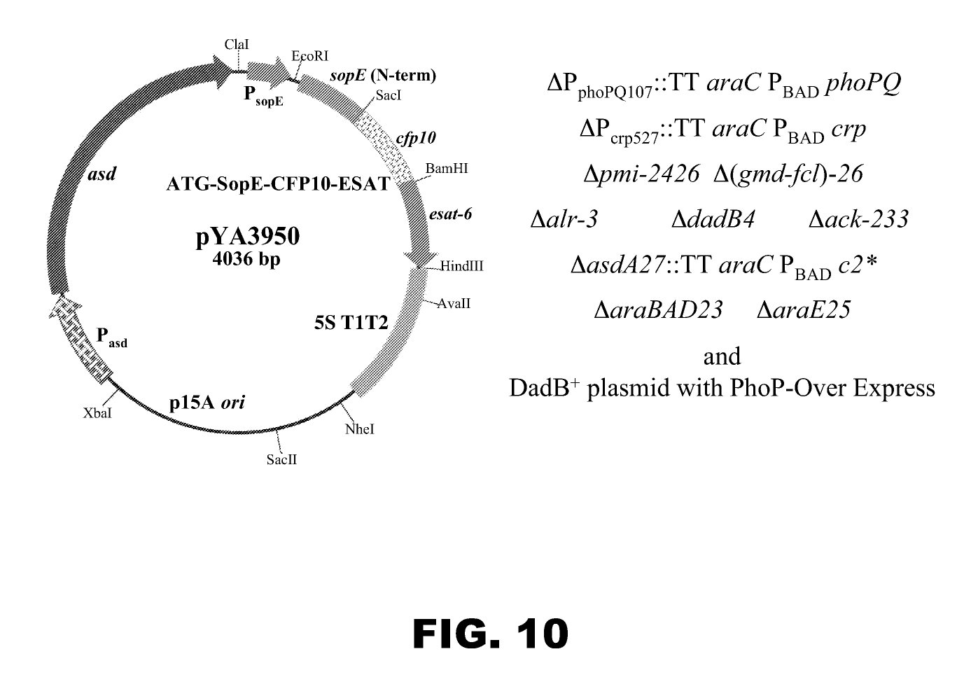

Alternatively, if a cytotoxic T lymphocyte (CTL) response is desired, then a Type III secretion system may be used (FIG. 10). Type III secretion systems are known in the art. This type of antigen delivery system delivers the antigen to the cytoplasm of cells in the host to enhance induction of CTL responses. This method of delivery is used for inducing cellular immunity to viral, parasite and bacterial pathogens, such as Mycobacterium tuberculosis.

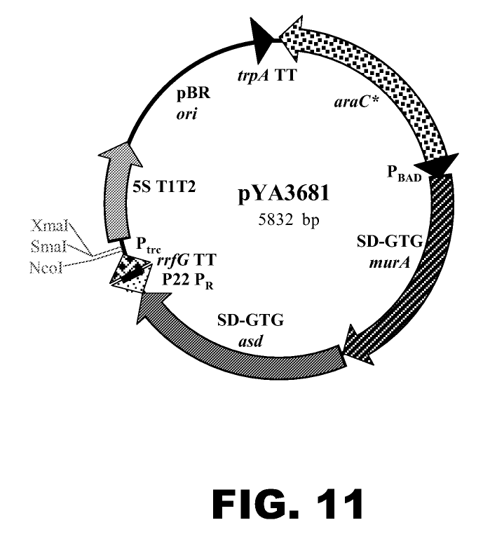

Yet another type of antigen delivery strategy that may be used is regulated delayed lysis of a bacterium in vivo to release protein antigen(s) and/or viral proteins. The viral proteins may include viral core particles with or without epitope fusion. Regulated antigen delivery systems are known in the art. See, for example, U.S. Pat. No. 6,780,405, hereby incorporated by reference in its entirety, and FIG. 11.

### (c) Inhibing Recombination

Although extrachromosomal vectors, such as plasmids, may be designed with unique nucleotide sequences, there is some potential for vector-vector recombination to occur that might lead to deletion of and/or alterations in one or more nucleic acid sequences encoding an antigen of interest. This could potentially expose a host to unintended antigens. Accordingly, in some embodiments, a recombinant bacterium of the invention may be deficient in one or more of the enzymes that catalyzes recombination between extrachromosomal vectors. If a bacterium comprises only a single extrachromosomal vector, then such mutations are not necessary. If two or more extrachromosomal vectors are used, however, then the recombinant bacterium may be modified so that one or more recombination enzymes known to catalyze vector-vector recombination are rendered non-functional.

In certain embodiments, the recombination enzymes do not participate in recombinations involving chromosomal nucleic acid sequences. For instance, the recombinant bacterium may comprise a ΔrecF and a ΔrecJ mutation. These mutations do not alter the virulence attributes of the recombinant bacterium, nor its ability to effectively colonize effector lymphoid tissues after immunization of a host. One of skill in the art will appreciate that other recombination enzymes known to catalyze vector-vector recombination but not to participate in recombinations involving chromosomal nucleic acid sequences may be targeted for deletion or mutation in addition to RecF and RecJ.

Alternatively, the recombinant bacterium may be modified by introducing a ΔrecA mutation that prevents all recombination, whether between vectors or chromosomal nucleic acid sequences. A recombinant bacterium with a ΔrecA mutation is also attenuated. A ΔrecA mutation, however, may diminish a bacterium's ability to colonize effector lymphoid tissues after oral or intranasal immunization. To counter this, a recombinant bacterium may be constructed with a ΔP_{recA}:: araC P_{BAD} recA insertion-deletion mutation so that expression of the RecA recombination enzyme is dependent on the presense of arabinose in the growth medium. In this system, the recombinant bacterium with the ΔP_{recA}:: araC P_{BAD} recA mutation is grown in medium devoid of arabinose to preclude vector-vector recombination. Then, just prior to administration of the recombinant bacterium to a host, arabinose may be supplied to enable expression of the nucleic acid encoding the RecA enzyme. This allows the recombinant bacterium to efficiently colonize effector lymphoid tissues. However, since there is no arabinose present in animal or human host tissues, the RecA enzyme will be depleted by cell division and the absence of recombination in vivo can be restored. Such a strategy may be used in addition to, or in place of, using ΔrecF and ΔrecJ mutations.

### (d) Attenuation

In each of the above embodiments, a recombinant bacterium of the invention may also be attenuated. “Attenuated” refers to the state of the bacterium wherein the bacterium has been weakened from its wild type fitness by some form of recombinant or physical manipulation. This includes altering the genotype of the bacterium to reduce ability to cause disease. However, the bacterium's ability to colonize the gut (in the case of Salmonella) and induce immune responses are preferably not substantially compromised.

Methods of attenuation are known in the art. For instance, attenuation may be accomplished by altering (e.g., deleting) native nucleic acid sequences found in the wild type bacterium. For instance, if the bacterium is Salmonella, non-limiting examples of nucleic acid sequences which may be used for attenuation may include: a pab nucleic acid sequence, a pur nucleic acid sequence, an aro nucleic acid sequence, asd, a dap nucleic acid sequence, nadA, pncB, galE, pmi, fur, ompR, htrA, hemA, cdt, cya, crp, dam, phoP, phoQ, rfc, poxA, galU, mviA, sodC, recA, ssrA, sirA, inv, hilA, rpoS, rpoE, flgM, tonB, slyA, and any combination thereof. Exemplary attenuating mutations may be aroA, aroC, aroD, cdt, cya, crp, phoP, phoQ, ompR, galE, and htrA.

In certain embodiments, the above nucleic acid sequences may be placed under the control of a sugar regulated promoter such that the sugar is added during in vitro growth of the recombinant bacterium, and the sugar is substantially absent within an animal or human host. The cessation in transcription of the nucleic acid sequences listed above would then result in attenuation and the inability of the recombinant bacterium to induce disease symptoms.

In another embodiment, the recombinant bacterium may contain one and in some embodiments, more than one, deletion and/or deletion-insertion mutations present in the Salmonella strains listed in Table 1 above.

### II. Vaccine Compositions and Methods of Administration

A recombinant bacterium of the invention may be administered to a host as a vaccine composition. As used herein, a vaccine composition is a composition designed to illicit an immune response to the recombinant bacterium. In an exemplary embodiment, the immune response is protective, as described above. Immune responses to antigens are well studied and widely reported. A survey of immunology is given in Paul, Ed. (1999), Fundamental Immunology, fourth ed., Philadelphia: Lippincott-Raven, Sites et al., Basic and Clinical Immunology (Lange Medical Books, Los Altos, Calif., 1994), and Orga et al., Handbook of Mucosal Immunology (Academic Press, San Diego, Calif., 1994). Mucosal immunity is also described by Ogra et al., Eds. (1999), Mucosal Immunology, second ed., Academic Press, San Diego.

Vaccine compositions of the present invention may be administered to any host capable of mounting an immune response. Such hosts may include all vertebrates, for example, mammals, including domestic animals, agricultural animals, laboratory animals, and humans, and various species of birds, including domestic birds and birds of agricultural importance. Preferably, the host is a warm-blooded animal. The vaccine may be administered as a prophylactic or for treatment purposes.

In exemplary embodiments, the recombinant bacterium is alive when administered to a host in a vaccine composition of the invention. Suitable vaccine composition formulations and methods of administration are detailed below.

### (a) Vaccine Composition

A vaccine composition comprising a recombinant bacterium of the invention may optionally comprise one or more possible additives, such as carriers, preservatives, stabilizers, adjuvants, and other substances.

In one embodiment, the vaccine comprises an adjuvant. Adjuvants, such as aluminum hydroxide or aluminum phosphate, are optionally added to increase the ability of the vaccine to trigger, enhance, or prolong an immune response. In some embodiments, the use of a live attenuated recombinant bacterium may act as a natural adjuvant. The vaccine compositions may further comprise additional components known in the art to improve the immune response to a vaccine, such as adjuvants, T cell co-stimulatory molecules, or antibodies, such as anti-CTLA4. Additional materials, such as cytokines, chemokines, and bacterial nucleic acid sequences naturally found in bacteria, like CpG, are also potential vaccine adjuvants.

In another embodiment, the vaccine may comprise a pharmaceutical carrier (or excipient). Such a carrier may be any solvent or solid material for encapsulation that is non-toxic to the inoculated host and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients as well as formulations for parenteral and nonparenteral drug delivery are set forth in Remington's Pharmaceutical Sciences 19th Ed. Mack Publishing (1995). When used for administering via the bronchial tubes, the vaccine is preferably presented in the form of an aerosol.

Care should be taken when using additives so that the live recombinant bacterium does not get killed or have its ability to effectively colonize lymphoid tissues such as the GALT, NALT and BALT affected by the use of additives. Stabilizers, such as lactose or monosodium glutamate (MSG), may be added to stabilize the vaccine formulation against a variety of conditions, such as temperature variations or a freeze-drying process.

The dosages of a vaccine composition of the invention can and will vary depending on the recombinant bacterium, the antigen, and the intended host, as will be appreciated by one of skill in the art. Generally speaking, the dosage need only be sufficient to elicit a protective immune response in a majority of hosts. Routine experimentation may readily establish the required dosage. Typical initial dosages of vaccine for oral administration could be about 1×10⁷ to 1×10¹⁰ CFU depending upon the age of the host to be immunized. Administering multiple dosages can also be used as needed to provide the desired level of protective immunity.

### (b) Methods of Administration

In order to stimulate a preferred response of the GALT, NALT, or BALT cells, administration of the vaccine composition directly into the gut, nasopharynx, or bronchus is preferred, such as by oral administration, intranasal administration, gastric intubation or in the form of aerosols, although other methods of administering the recombinant bacterium, such as intravenous, intramuscular, intradermal, intraperitoneal, intralymphatic, percutaneous, scarification, subcutaneous injection or intramammary, intrapenial, intrarectal, vaginal administration, other parenteral routes, or any other route relevant for an infectious disease is possible.

In embodiments where these compositions are administration by injection (e.g., intraperitoneally, intravenously, subcutaneously, intramuscularly, etc.), the compositions are preferably combined with pharmaceutically acceptable vehicles such as saline, Ringer's solution, dextrose solution, and the like.

In an exemplary embodiment, recombinant bacterium may be administered orally. Oral administration of a composition comprising a recombinant bacterium allows for greater ease in disseminating vaccine compositions for infectious agents to a large number of people in need thereof, for example, in Third World countries or during times of biological warfare. In addition, oral administration allows for attachment of the bacterium to, and invasion of, the gut-associated lymphoid tissues (GALT or Peyer's patches) and/or effective colonization of the mesenteric lymph nodes, liver, and spleen. This route of administration thus enhances the induction of mucosal immune responses as well as systemic and cellular immune responses.

### III. Kits

The invention also encompasses kits comprising any one of the compositions above in a suitable aliquot for vaccinating a host in need thereof. In one embodiment, the kit further comprises instructions for use. In other embodiments, the composition is lyophilized such that the addition of a hydrating agent (e.g., buffered saline) reconstitutes the composition to generate a vaccine composition ready to administer, preferably orally.

### IV. Methods of Use

A further aspect of the invention encompasses methods of using a recombinant bacterium of the invention. For instance, in one embodiment the invention provides a method for modulating a host's immune system. The method comprises administering to the host an effective amount of a composition comprising a recombinant bacterium of the invention, wherein the bacterium comprises at least one extrachromosomal vector, as described herein, encoding one or more antigens. One of skill in the art will appreciate that an effective amount of a composition is an amount that will generate the desired immune response (e.g., mucosal, humoral or cellular). Methods of monitoring a host's immune response are well-known to physicians and other skilled practitioners. For instance, assays such as ELISA, and ELISPOT may be used. Effectiveness may be determined by monitoring the amount of the antigen of interest remaining in the host, or by measuring a decrease in disease incidence caused by a given pathogen in a host. For certain pathogens, cultures or swabs taken as biological samples from a host may be used to monitor the existence or amount of pathogen in the individual.

In another embodiment, the invention provides a method for eliciting an immune response against an antigen in a host. The method comprises administering to the host an effective amount of a composition comprising a recombinant bacterium of the invention, wherein the bacterium comprises at least one extrachromosomal vector, as described herein, that encodes an antigen. In an exemplary embodiment, the bacterium is attenuated. In another exemplary embodiment, the expression of the nucleic acid encoding the antigen may be regulated such that the nucleic acid is not expressed in vitro. In a further exemplary embodiment, the recombinant bacterium is deficient in one or more enzymes that catalyzes recombination between extrachromosomal vectors.

In yet another embodiment, the invention provides a method for eliciting an immune response against multiple antigens in a host. The method comprises administering to the host an effective amount of a composition comprising a recombinant bacterium as described herein. In an exemplary embodiment, the bacterium is attenuated. In another exemplary embodiment, the expression of the nucleic acid(s) encoding the multiple antigens may be regulated such that the nucleic acid(s) is not expressed in vitro. In a further exemplary embodiment, the recombinant bacterium is deficient in one or more enzymes that catalyzes recombination between extrachromosomal vectors.

In still another embodiment, a recombinant bacterium of the invention may be used in a method for eliciting an immune response against a pathogen in an individual in need thereof. The method comprises administrating to the host an effective amount of a composition comprising a recombinant bacterium as described herein. In a further embodiment, a recombinant bacterium described herein may be used in a method for ameliorating one or more symptoms of an infectious disease in a host in need thereof. The method comprises administering an effective amount of a composition comprising a recombinant bacterium as described herein.

A recombinant bacterium of the invention also may be used in a method for easier vaccine manufacturing. The recombinant bacteria can be readily grown in batches and processed. Since a bacterium of this invention is not dependent on antibiotics, the cost of producing vaccines based on this type of recombinant bacterium is reduced.

### DEFINITIONS

The term “altered,” as used herein, refers to any change in the nucleic acid sequence that results in the nucleic acid sequence not being expressed. In an exemplary embodiment, the alteration results in the nucleic acid sequence not being expressed in a host. In one embodiment, the alteration is a deletion. In another embodiment, the alteration places an essential nucleic acid under the control of a regulatable promoter, such that the nucleic acid is not expressed in a host.

The term “native,” as used herein, refers to a biomolecule in form typically found in the strain a recombinant bacterium of the invention is derived from.

The term “operably linked,” as used herein, means that expression of a nucleic acid is under the control of a promoter with which it is spatially connected. A promoter may be positioned 5′ (upstream) of a nucleic acid under its control. The distance between the promoter and a nucleic acid may be approximately the same as the distance between that promoter and the nucleic acid it controls in the nucleic acid from which the promoter is derived. As is known in the art, variation in this distance may be accommodated without loss of promoter function.

The term “promoter”, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same.

The term “virulence,” as used here, refers to the ability of the recombinant bacterium to infect a host.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

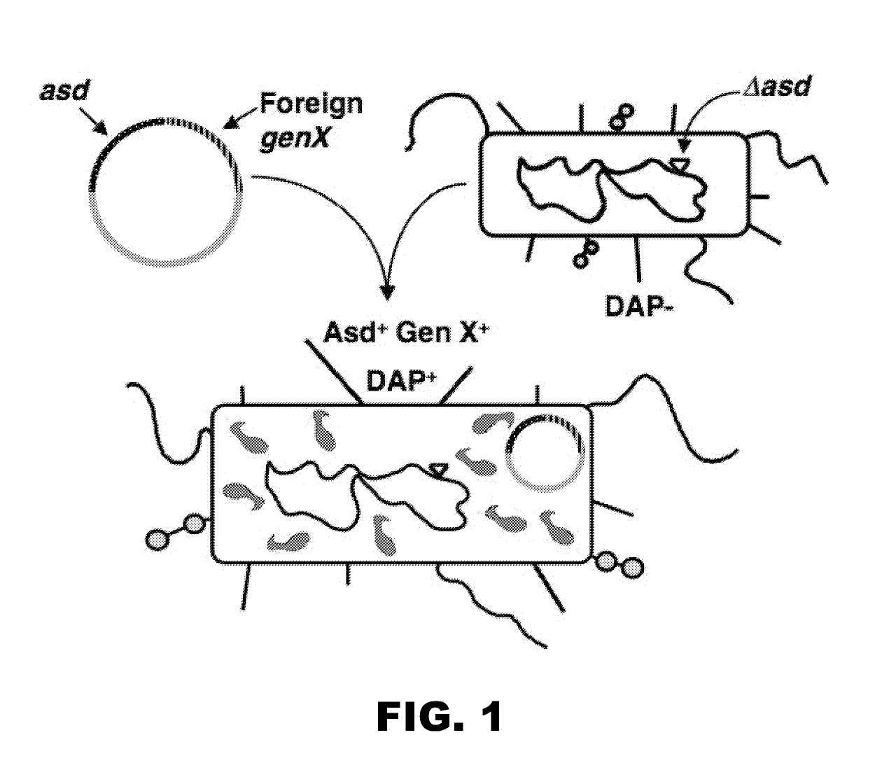

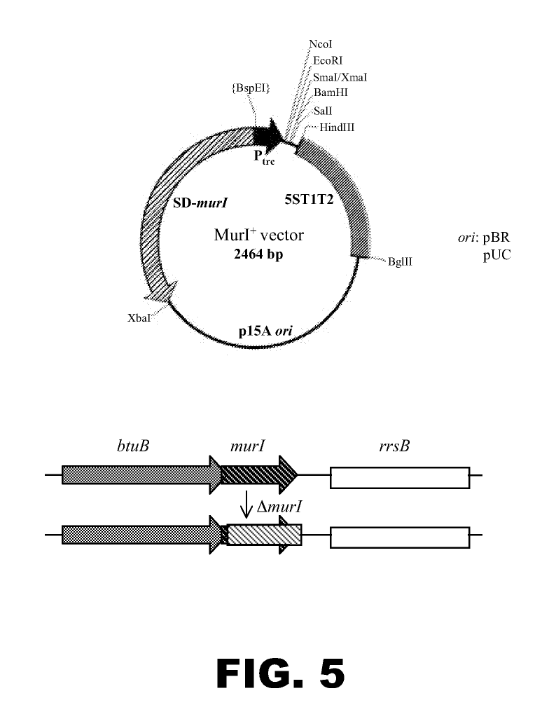

In this example, AsdA⁺ plasmids were constructed to complement a ΔasdA mutation in E. coli strains such as χ6212 and χ6097 and in Salmonella strains such as χ8276 and χ8958 (Table 1). The ΔasdA mutation, eliminates the ability to produce aspartate semialdehyde dehydrogenase, an enzyme essential for the synthesis of diaminopimelic acid (DAP). FIG. 1 illustrates how the balanced-lethal plasmid system works with an asd deletion (Δ) mutation. One or more antigens of interest, such as a protective antigen, were cloned into various AsdA⁺ plasmids using the multiple cloning site on each Asd⁺ plasmid. Thus, pYA3493 (FIG. 5) and pYA3620 (FIG. 6) were used to clone a DNA sequence encoding amino acids 3 to 285 including the alpha-helical portion of the PspA protein from Streptococcus pneumoniae strain Rx1. These plasmid constructs (pYA4088 and pYA3802, Table 2) were first introduced into χ6212 and evaluated, using western blot analysis, for stability, and synthesis and secretion of the recombinant PspA antigens of the appropriate size. These plasmids were then introduced into Salmonella strain χ8276, which is deficient in Asd. The AsdA⁺ plasmid synthesizes the Asd enzyme required to complement the AsdA⁻Salmonella and also synthesizes the antigen of interest (indicated in FIG. 1 as GenX). As such, the result is a Salmonella strain that synthesizes Asd, and consequently, can synthesize DAP, and also synthesizes the foreign antigen of interest, in this case PspA.

The χ8276 derivatives with pYA4088 and pYA3802 were then evaluated for both rate of growth, stability in maintenance, and production of PspA over 50 generations of growth in LB broth medium supplemented with DAP (a non-selective condition). Passing these tests, pYA4088 and pYA3802 were introduced into attenuated Salmonella vaccine strains χ8133 and χ9088 (Table 1). These strains were used to orally immunize 8-week old female BALB/c mice and sera and vaginal washings were collected over a period of 12 weeks at two-week intervals. Antibody titers in sera and vaginal secretions to the PspA antigen were higher than to Salmonella LPS and OMP antigens. These mice were protected against intraperitoneal challenge with 250 times a lethal dose of S. pneumoniae WU2. Protective immunity was also transferred in the form of sera and spleen cells to confer protective immunity to WU challenge to naive unimmunized mice. Many other protective antigens listed in Table 3 have been cloned into the Asd⁺ vectors depicted in FIGS. 2, 6, 7, 8, and 9 and these are listed in Table 2. They have then ultimately been introduced into Salmonella vaccine strains listed in Table 1 and evaluated for colonizing ability, ability to induce antibody and cellular immune responses to the expressed antigens in mice in the case of antigens from S. pneumoniae, M. tuberculosis, Y. pestis and influenza virus. In evaluating vaccines expressing S. pneumoniae and Y. pestis antigens, we have demonstrated protection to challenge with virulent wild-type S. pneumoniae and Y. pestis strains.

### Example 2

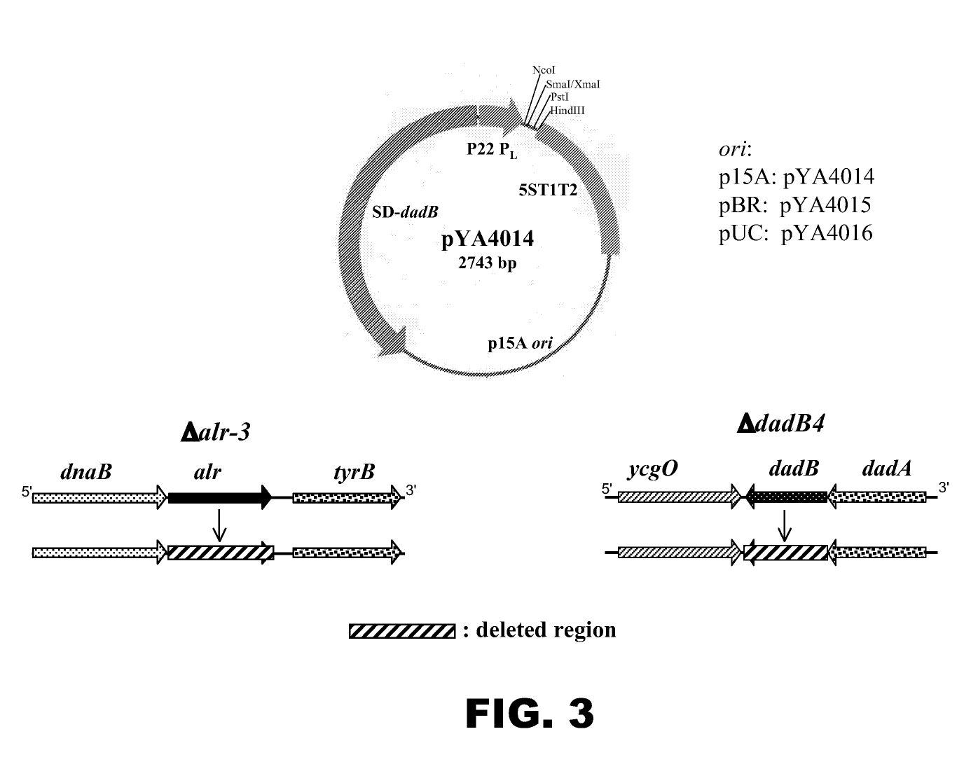

In this example, Salmonella strains with Δalr and ΔdadB mutations were used to eliminate the Salmonella's ability to produce two different alanine racemases, enzymes essential for the synthesis of D-alanine (another unique essential constituent of the peptidoglycan layer of the bacterial cell wall). The DadB⁺ plasmids with different origins of replication were used as shown in FIG. 3 to express foreign antigens of interest. In one case, the IpxE gene from Francisella tularensis was cloned into the DadB⁺ vector pYA4014 (FIG. 3) to yield pYA4021 and the S. typhimurium pagL gene into the Asd⁺ vector pYA3337 (FIG. 2) to yield pYA4019. Both pYA4021 and pYA4019 were then introduced into χ9040 (Table 1) to evaluate structure, function and toxicity of lipid A. The lipid A of the Salmonella LPS is the endotoxin and co-expression of the PagL and LpxE proteins was determined to render lipid A non-toxic but to retain abilities to interact with murine and human TLR4 to recruit innate immunity. Using the recombinant χ9040 strain also enabled evaluation of virulence, colonizing ability and immunogenicity (in survivors). This discovery and findings have been used to design Salmonella vaccine strains with regulated chromosomal copies of IpxE and pagL genes expressing high levels of PagL and LpxE to engender these vaccines safe and non-toxic to administer to newborns. Thus, the ability to stably maintain two different plasmids in the same Salmonella strain enabled development of a safer improved vaccine strain for newborns and infants.

### Example 3

In this example, the phoP gene has been cloned into the DadB⁺ vectors pYA4014, pYA4015 and pYA4016 (FIG. 3) to yield pYA3833, pYA3861 and pYA3862, respectively (Table 2). In all cases, the expression of the phoP gene is under the control of the C2 repressor specified by various ΔasdA::TT araC P_{BAD} c2 deletion-insertion mutations as present in χ9048, χ9291, χ9292, χ9340, χ9388, and χ9389 (Table I). When any of these strains possess pYA3833, pYA3861 or pYA3862, and are grown in the presence of arabinose (media is also supplemented with DAP), the C2 repressor protein is synthesized and the PhoP protein is not synthesized. χ9291 and χ9292 also have the ΔrecF and ΔrecJ mutations to minimize if not prevent plasmid-plasmid recombination.

To facilitate animal studies, these strains were transformed with the compatible Asd⁺ plasmid pYA3337 (FIG. 2). After oral inoculation of mice, arabinose is absent and C2 repressor ceases to be synthesized and is diluted out at each cell division. This results in increasing synthesis of the PhoP protein, which is totally attenuating and has been shown to enhance induction of cell-mediated immune responses such as CTL responses. Thus, the Asd⁺ plasmid pYA3950 (FIG. 10) was introduced into χ9388 and χ9389 along with either pYA4015 or pYA4016 to examine induction of cell-mediated immune responses to the M tuberculosis antigens CFP-10 and ESAT-6.

Both of these antigens are known to induce CTL immune responses in both mice and humans. The pYA3950 vector is designed for delivery of the SopE-CFP-10-ESAT-6 fusion via the Type III Secretion System (TTSS) such that the fusion is delivered to the cytosol of cells within the immunized individual. The SopE component not only escorts the CFP-10-ESAT-6 antigens through the TTSS needle to the cytosol but is rapidly ubiquinated to facilitate antigen traffic to the proteosome for rapid class I MHC presentation. All of these features and especially over production of PhoP enhance CMI responses.

### Example 4

In this example, Salmonella was attenuated using the ΔP_{murA}::TT araC P_{BAD} murA deletion-insertion mutation and various ΔasdA mutations to enable use of the regulated delayed lysis in vivo vector pYA3681 (FIG. 11). Thus, pYA3681 was engineered by insertion of a sequence encoding the alpha-helical portion of the Rx1 PspA antigen to yield pYA3712 (Table 2) and this plasmid was introduced into Salmonella vaccine strains designed for regulated delayed lysis to deliver protective antigens. These include strains χ8937, χ9412, χ9413, χ9420 and χ9514 (Table I). The recombinant pYA3712 plasmid was thus introduced into χ8937 and the recombinant vaccine used to orally immunize female BALB/c mice. Excellent immune responses to the PspA antigen were induced and mice were protected against challenge with virulent S. pneumoniae WU2.

### Example 5

Other recombinant plasmids specifying Type II (FIGS. 6, 7, 8 and 9) or Type III (FIG. 10) secretion of protective antigens or for regulated delayed lysis and antigen delivery (FIG. 11) have been (Table 2) or are generated and introduced into the hundreds of candidate attenuated Salmonella vaccine strains constructed, many of which are listed in Table 1. In other cases, strains have been endowed with abilities to modulate lipid A toxicity, to enhance recruitment of innate immunity and to enhance cell-mediated immunity. In these cases, Asd⁺, DadB⁺, MurA⁺, and Mur¹⁺ plasmids are used to express antigens or enzymes that are beneficial in modulating the performance of vaccine strains or in deducing how to improve vaccine strains.

Many of these recombinant plasmids and the vectors from which they were derived are listed in Table 2 and some of the antigens cloned into these vectors are listed in Table 3. Table 1 lists many Salmonella strains suitable to evaluate stability, antigen production and immunogenicity in animals. Table 1 also lists the E. coli strains used in initial cloning and as suicide vector delivery stains to introduce recombinant plasmids into attenuated Salmonella vaccine strains.

### Example 6

In the forgoing Examples, the Salmonella strains all possessed chromosomal deletion mutations that blocked the ability to synthesize an essential constituent of the peptidoglycan layer of the cell wall. In the absence of such required nutrients imposed by the presence of the deletion mutations the bacterial cells outgrow their skins so-to-speak and die by lysis with liberation of their cell contents. In other cases, it is not possible to delete genes for synthesis of essential constituents when uptake of the required nutrient is not possible as in the case when the nutrient is phosphorylated. In these cases, genetic modifications were made to generate arabinose-regulated expression of essential genes such that viability is maintained as long as arabinose is present in the growth medium with death by lysis ensuing when arabinose is absent. When any of these mutational alterations in chromosomal genes, whether by deletion or by establishing a state of conditional lethality, are complimented by the presence of a vector with the wild-type DNA sequence encoding the gene that is missing or not synthesized by the chromosomal genetic alteration, a balanced-lethal vector-host state is established. In these cases, loss of the vector will lead to death by lysis with liberation of cell contents if such vector loss occurs in an environment in which required essential nutrients or arabinose are absent such as is the case in tissues within an animal or human host.

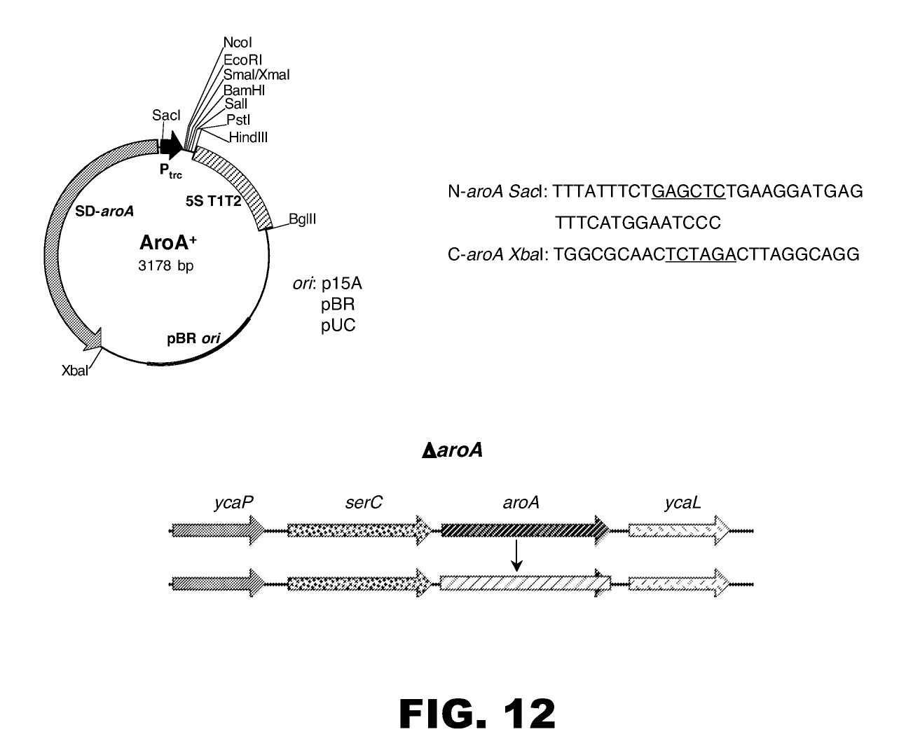

In addition to essential genes encoding enzymes that are necessary for bacterial cell survival, there are other genes essential for survival in certain environments such as in tissues of an animal or human host. Pathogens such as Salmonella must maintain the ability to synthesize nutrients not present within the animal or human host in order to be a successful pathogen. It therefore follows that mutant strains of a pathogen that are unable to synthesize nutrients, such as essential amino acids (e.g. isoleucine, valine or tryptophan) or vitamins (e.g. p-aminobenzoic acid) or purines (e.g. adenine) would display reduced virulence and be attenuated. The avirulence of such mutants is well known and thus these metabolic genes can be construed to be essential for virulence. It therefore follows, that vectors possessing the wild-type sequence for these metabolic virulence genes should complement mutational alterations including deletions of these metabolic genes in the bacterial chromosome and restore virulence to the mutant strains. This is also known to be true. In the design of vectors with the wild-type sequence for metabolic virulence genes it is possible to insert regulatable promoters followed by multiple cloning sites to facilitate insertion of DNA sequences encoding proteins or antigens of interest followed by a transcription termination sequence and with a specific origin of replication to give a desired number of vector copies per cell. Such recombinant constructions will be desirable components of live recombinant bacterial vaccine formulations and enable synthesis of desired proteins or antigens and delivery to an animal or human host so as to induce an immune response to the synthesized and delivered antigen, for example. FIGS. 12, 13, 14, 15, and 16 depict plasmid vectors with the Lacl repressible P_{trc} promoter followed by a multiple cloning site to enable insertion of sequences encoding proteins or antigens of interest followed by the 5S rRNA transcription terminator and the pBR on and with the wild-type aroA, aroC, aroD, ilvC and ilvE genes to complement chromosomal ΔaroA, ΔaroC, ΔaroD, ΔilvC and ΔilvE mutations, respectively. The defined deletion mutations can readily be introduced into vaccine strains listed in Table 1 using suicide vector technologies and generalized transduction. The inclusion of these additional vector systems diversifies the options in design and construction of recombinant attenuated Salmonella vaccines capable of synthesis and delivery, including by Type II or Type III secretion, or by regulated lysis, of multiple protective antigens thus enhancing means to develop more efficacious vaccines to prevent or treat infectious diseases of animals and humans.

### REFERENCES

1. Brown, E. D., E. I. Vivas, C. T. Walsh, and R. Kolter. 1995. MurA (MurZ), the enzyme that catalyzes the first committed step in peptidoglycan biosynthesis, is essential in Escherichia coli. J. Bacteriol. 177:4194-4197
2. Buchmeier, N. A., S. J. Libby, Y. Xu, P. C. Loewen, J. Switala, D. G. Guiney, and F. C. Fang. 1995. DNA repair is more important than catalase for Salmonella virulence in mice. J. Clin. Invest. 95:1047-1053
3. Curtiss, R., III. 1990. Attenuated Salmonella strains as live vectors for the expression of foreign antigens, p. 715-740. In G. C. Woodrow and M. M. Levine (ed.), New Generation Vaccines. Marcel Dekker, New York, N.Y.
4. Curtiss, R., III, T. Doggett, A. Nayak, and J. Srinivasan. 1996. Strategies for the use of live recombinant avirulent bacterial vaccines for mucosal immunization, p. 499-511. In H. Kiyono and M. F. Kagnoff (ed.), Essentials of Mucosal Immunology. Academic Press, San Diego.
5. Doublet, P., J. van Heijenoort, J. P. Bohin, and D. Mengin-Lecreulx. 1993. The murl gene of Escherichia coli is an essential gene that encodes a glutamate racemase activity. J. Bacteriol. 175:2970-2979
6. Galan, J. E., and P. J. Sansonetti. 1996. Molecular and ellular bases of Salmonella and Shigella interactions with host cells, p. 2757-2773. In F. C. Neidhardt, R. Curtiss III, J. L. Ingraham, E. C. C. Lin, K. B. Low, B. Magasanik, W. S. Reznikoff, M. Riley, M. Schaechter, and H. E. Umbarger (ed.), Escherichia coli and Salmonella. Cellular and Molecular Biology, vol. 1. ASM Press, Washington, D.C.
7. Garzon, A., C. R. Beuzon, M. J. Mahan, and J. Casadesus. 1996. recB recJ mutants of Salmonella typhimurium are deficient in transductional recombination, DNA repair and plasmid maintenance. Mol. Gen. Genet. 250:570-580
8. Ivancic-Bace, I., E. Salaj-Smic, and K. Brcic-Kostic. 2005. Effects of recJ, recQ, and recFOR mutations on recombination in nuclease-deficient recB recD double mutants of Escherichia coli. J. Bacteriol. 187:1350-1356
9. Kang, H. Y., J. Srinivasan, and R. Curtiss III. 2002. Immune responses to recombinant pneumococcal PspA antigen delivered by live attenuated Salmonella enterica serovar typhimurium vaccine. Infect. Immun. 70:1739-1749
10. Lobocka, M., J. Hennig, J. Wild, and T. Klopotowski. 1994. Organization and expression of the Escherichia coli K-12 dad operon encoding the smaller subunit of D-amino acid dehydrogenase and the catabolic alanine racemase. J. Bacteriol. 176:1500-1510
11. Schodel, F. 1992. Prospects for oral vaccination using recombinant bacteria expressing viral epitopes. Adv. Virus Res. 41:409-446
12. Wasserman, S. A., C. T. Walsh, and D. Botstein. 1983. Two alanine racemase genes in Salmonella typhimurium that differ in structure and function. J. Bacteriol. 153:1439-1450

1. A recombinant bacterium, wherein the bacterium comprises:
a. a first chromosomally encoded essential nucleic acid sequence, wherein the first essential nucleic acid sequence is altered so that it is not expressed;
b. a second chromosomally encoded essential nucleic acid sequence, wherein the second essential nucleic acid sequence is altered so that it is not expressed;
c. a first extrachromosomal vector, the vector comprising a nucleic acid sequence, that when expressed, substantially functions as the first essential nucleic acid sequence; and
d. a second extrachromosomal vector, the vector comprising a nucleic acid sequence, that when expressed, substantially functions as the second essential nucleic acid sequence.

2. The recombinant bacterium of claim 1, wherein the first and second extrachromosomal vectors each further comprise a nucleic acid sequence encoding at least one antigen.

3. The recombinant bacterium of claim 1, wherein the first and second extrachromosomal vectors each further comprise a nucleic acid sequence encoding at least two antigens.

4. The recombinant bacterium of claim 1, wherein the first and/or second essential nucleic acid sequence is selected from the group consisting of a nucleic acid sequence encoding Alr, DadB, Dap, MurA, Murl, Asd, AroA, AroC, AroD, IlvC, and IlvE.

5. The recombinant bacterium of claim 1, wherein the altered first and/or second essential nucleic acid sequence is selected from the group consisting of a ΔasdA mutation, a Δdap mutation, a ΔdadB mutation with a Δalr mutation, a ΔP_{murA}::TT araC P_{BAD} murA deletion-insertion mutation, a Δmurl mutation, a ΔaroA mutation, a ΔaroC mutation, a ΔaroD mutation, a ΔilvC mutation, and a ΔilvE mutation.

6. The recombinant bacterium of claim 1, wherein the recombinant bacterium is deficient in recombination.

7. The recombinant bacterium of claim 1, wherein the recombinant bacterium is attenuated.

8. The recombinant bacterium of claim 1, wherein the bacterium comprises a third chromosomally encoded essential nucleic acid sequence, wherein the third essential nucleic acid sequence is altered so that it is not expressed, and a third extrachromosomal vector, the vector comprising a nucleic acid sequence, that when expressed, substantially functions as the third essential nucleic acid sequence.

9. The recombinant bacterium of claim 8, wherein the third extrachromosomal vector further comprises a nucleic acid sequence encoding at least one antigen.

10. The recombinant bacterium of claim 8, wherein the first, second, and/or third essential nucleic acid sequence is selected from the group consisting of a nucleic acid sequence encoding Alr, DadB, Dap, MurA, Murl, Asd, AroA, AroC, AroD, IlvC, and IlvE.

11. (canceled)

12. (canceled)

13. (canceled)

14. The recombinant bacterium of claim 1, wherein the bacterium comprises a fourth chromosomally encoded essential nucleic acid sequence, wherein the fourth essential nucleic acid sequence is altered so that it is not expressed, and a fourth extrachromosomal vector, the vector comprising a nucleic acid sequence, that when expressed, substantially functions as the fourth essential nucleic acid sequence.

15. The recombinant bacterium of claim 14, wherein the fourth extrachromosomal vector further comprises a nucleic acid sequence encoding at least one antigen.

16. The recombinant bacterium of claim 14, wherein the first, second, third, and/or fourth essential nucleic acid sequence is selected from the group consisting of a nucleic acid sequence encoding Alr, DadB, Dap, MurA, Murl, Asd, AroA, AroC, AroD, IlvC, and IlvE.

17. (canceled)

18. (canceled)

19. (canceled)

20. A recombinant bacterium, wherein the bacterium comprises:
a. a chromosomally encoded essential nucleic acid sequence whose expression is necessary for a metabolic activity essential for virulence, wherein the essential nucleic acid sequence is altered so that it is not expressed, and
b. an extrachromosomal vector, the vector comprising a nucleic acid sequence, that when expressed, substantially functions as the essential nucleic acid sequence.

21. The recombinant bacterium of claim 20, wherein the extrachromosomal vector comprises a nucleic acid sequence encoding at least one antigen.

22. The recombinant bacterium of claim 21, wherein the extrachromosomal vector comprises a nucleic acid sequence encoding at least two antigens.

23. The recombinant bacterium of claim 20, wherein the essential nucleic acid sequence is selected from the group consisting of a nucleic acid sequence encoding AroA, AroC, AroD, IlvC, and IlvE.

24. The recombinant bacterium of claim 20, wherein the altered essential nucleic acid sequence is selected from the group consisting of a ΔaroA mutation, a ΔaroC mutation, a ΔaroD mutation, a ΔilvC mutation, and a ΔilvE mutation.

25. The recombinant bacterium of claim 20, wherein the recombinant bacterium is deficient in recombination.

26. The recombinant bacterium of claim 20, wherein the recombinant bacterium is attenuated.


SEQUENCE LISTING (19 sequences)

SEQ ID NO: 1 - DNA, 168 bp, Artificial Sequence
  SALMONELLA
aggaaacagaccatgagtattcaacatttccgtgtcgcccttattcccttttttgcggcattttggcttcctgtttttgctcacccagaaacgctggtgaaagtaaaagatgctgaagaattcgcaattcccggggatccgtcgacctgcagccaagctcccaagctt

SEQ ID NO: 2 - protein, 35 aa, Artificial Sequence
  SALMONELLA
MSIQHFRVALIPFFAAFCLPVFAHPETLVKVKDAE

SEQ ID NO: 3 - DNA, 290 bp, Artificial Sequence
  SALMONELLA
gctgttgacaattaatcatccggctcgtataatgtgtggaattgtgagcggataacaatttcacacaggaaacagaccatgagtattcaacatttccgtgtcgcccttattcccttttttgcggcattttgccttcctgtttttgctcacccagaaacgctggtgaaagtaaaagatgctgaagaattcgcaattcccggggatccgtcgacctgcaggcaactatggatgaacgaaatagacagatcgctgagataggtgcctcactgattaagcattggtaaaagctt

SEQ ID NO: 4 - protein, 35 aa, Artificial Sequence
  SALMONELLA
MSIQHFRVALIPFFAAFCLPVFAHPTLVKKVKDAE

SEQ ID NO: 5 - protein, 21 aa, Artificial Sequence
  SALMONELLA
ATMDERNRQIAEIGASLIKHW

SEQ ID NO: 6 - DNA, 205 bp, Artificial Sequence
  SALMONELLA
aatgagctgttgacaattaatcatccggctcgtataatgtgtgggtggaattgtgagcggataacaatttcacacaggaaacagaccatgaaaaagacagctatcgcgattgcagtggcactggctggtttcgctaccgtagcgcaggccgcggaattccgaaattcccggggatccgtcgacctgcagccaagctcccaagctt

SEQ ID NO: 7 - protein, 22 aa, Artificial Sequence
  SALMONELLA
MKKTAIAIAVALAGFATVAQAA

SEQ ID NO: 8 - DNA, 210 bp, Artificial Sequence
  SALMONELLA
aatgagctgttgacaattaatcatccggctcgtataatgtgtgggtggaattgtgagcggataacaatttcacacaggaaacagaccatgaaacaaagcactattgcactggcactgctgctgccgctgctgtttacccctgtgaccaaagcccgtaccccagaaatgaacccggggatccgtcgacctgcagccaagctcccaagcttg

SEQ ID NO: 9 - protein, 27 aa, Artificial Sequence
  SALMONELLA
MKQSTIALALLPLLFTPVTKARTPEMN

SEQ ID NO: 10 - DNA, 40 bp, Artificial Sequence
  SALMONELLA
tttatttctgagctctgaaggatgagtttcatggaatccc

SEQ ID NO: 11 - DNA, 25 bp, Artificial Sequence
  SALMONELLA
tggcgcaactctagacttaggcagg

SEQ ID NO: 12 - DNA, 43 bp, Artificial Sequence
  SALMONELLA
cgcatccggaggaggagccgaatggcaggaaacacaattggac

SEQ ID NO: 13 - DNA, 36 bp, Artificial Sequence
  SALMONELLA
gaatctagattaccagcgtggaatctctgtctttac

SEQ ID NO: 14 - DNA, 33 bp, Artificial Sequence
  SALMONELLA
cgaatccggaatgaaggataccaaatgaaaacc

SEQ ID NO: 15 - DNA, 34 bp, Artificial Sequence
  SALMONELLA
agatctagattacgcctggtgcaatatagttaat

SEQ ID NO: 16 - DNA, 49 bp, Artificial Sequence
  SALMONELLA
cgcatccggaggaagccagaaatggctaactactttaatacactgaatc

SEQ ID NO: 17 - DNA, 36 bp, Artificial Sequence
  SALMONELLA
gaatctagatcaacccgctaccgcaatacgcttcat

SEQ ID NO: 18 - DNA, 35 bp, Artificial Sequence
  SALMONELLA
ccgctcgagaggaagaaaaatgacgacgaaaaaag

SEQ ID NO: 19 - DNA, 34 bp, Artificial Sequence
  SALMONELLA
gaatctagattattatgaatttaccggatccaac